(19) United States
(12) Patent Application Publication
(10) Pub. No.: US 2022/0228211 A1
(43) Pub. Date: Jul. 21, 2022

(54) POLYMER BASED CELLULAR LABELING, BARCODING AND ASSEMBLY

(71) Applicant: CHILDREN'S HOSPITAL MEDICAL CENTER, Cincinnati, OH (US)

(72) Inventors: Takanori Takebe, Cincinatti, OH (US); Andrew Dunn, Worthington, OH (US)

(21) Appl. No.: 17/595,496

(22) PCT Filed: May 29, 2020
(86) PCT No.: PCT/US2020/035425

§ 371 Date: Nov. 17, 2021

Related U.S. Application Data
(60) Provisional application No. 62/855,448, filed on May 31, 2019.

(51) Int. Cl. C12Q 1/6881; C12Q 1/6806; C08F 283/06; C12N 15/10

(52) U.S. Cl. CPC C12Q 1/6881; C12Q 1/6806; C12Q 2600/16; C12N 15/1037; C08F 283/065

(57) ABSTRACT

Existing single cell analysis techniques are generally high-resolution but are limited in the number of possible different experimental conditions. Disclosed herein are compositions and methods for multiplexed barcoding of a heterogenous population of cells using cationic polymers for delivery of nucleic acid barcodes to a cell population.

### CROSS-REFERENCE TO RELATED APPLICATIONS

This application claims the benefit of priority of U.S. Provisional Patent Application No. 62/855,448, filed May 31, 2019, which is hereby expressly incorporated by reference in its entirety.

### REFERENCE TO SEQUENCE LISTING

The present application is being filed along with a Sequence Listing in electronic format. The Sequence Listing is provided as a file entitled CHMC63_022WOSeqListing.TXT, which was created and last modified on May 29, 2020, which is 1,305 bytes in size. The information in the electronic Sequence Listing is hereby incorporated by reference in its entirety.

### FIELD OF THE INVENTION

Aspects of the present disclosure relate generally to cell barcoding techniques. These techniques employ cationic polymers and synthesized nucleic acid molecules for efficient and inexpensive multiplexed barcoding.

### BACKGROUND

Single-cell genomic, transcriptomic, and proteomic analysis has revolutionized quantitative biology and applied medicine. Innovative techniques for high-throughput oligonucleotide sequencing have opened the path for an array of innovative strategies for the treatment and isolation of specific cell types and their subsequent investigation in downstream analysis. In single-cell applications, the current methodology relies on a single-cell labeling using an antibody-oligonucleotide pair which tags cell populations with unique molecular identifiers, acting as a molecular barcode. DNA oligonucleotides are covalently bound to the surface of specific antibodies; these antibodies act as a labeling mediator as oligonucleotides do not predominantly possess an innate ability to target and bind to cells or proteins of interest. Moreover, direct conjugation is required for each combination of antibody-oligonucleotide pairing. Labeling five populations of the same cell type with five different unique molecular identifies would require five separate conjugation reactions. This necessity of creating antibody-oligo pairs for every cell type can become laborious, costly, and time-consuming. Therefore, there is a present need for improved methods for cell labeling.

### SUMMARY

Some aspects of the present disclosure relate to methods of synthesizing a capped cationic polymer. In some embodiments, the methods comprise contacting poly(ethylene glycol) diacrylate monomers and 3-amino-1-propanol to form a poly(ethylene glycol) diacrylate/3-amino-1-propanol cationic polymer by Michael Addition, wherein the molar ratio of poly(ethylene glycol) diacrylate monomers to 3-amino-1-propanol is greater than 1, and wherein the cationic polymer is acrylate terminated and contacting the terminal acrylate groups of the cationic polymer with capping molecules comprising amine groups to form the capped cationic polymer by Michael Addition, wherein the capped cationic polymer does not comprise any acrylate groups. In some embodiments, the poly(ethylene glycol) diacrylate monomers and 3-amino-1-propanol of step (a) are further contacted with di(trimethylolpropane) tetraacrylate, wherein the addition of di(trimethylolpropane) tetraacrylate results in the formation of a branched poly(ethylene glycol) diacrylate/di(trimethylolpropane) tetraacrylate/3-amino-1-propanol cationic polymer comprising more than two terminal acrylate groups. In some embodiments, the capping molecules comprise one or more of 1,4-bis(3-aminopropyl)piperazine, spermine, polyethylenimine, or 2,2-dimethyl-1,3-propanediamine, or any combination thereof. In some embodiments, the molar ratio of poly(ethylene glycol) diacrylate monomers to 3-amino-1-propanol is 1.01:1, 1.02:1, 1.03:1, 1.04:1, 1.05:1, 1.06:1, 1.07:1, 1.08:1, 1.09:1, 1.1:1, 1.11:1, 1.12:1, 1.13:1, 1.14:1, or 1.15:1, or about 1.01:1, about 1.02:1, about 1.03:1, about 1.04:1, about 1.05:1, about 1.06:1, about 1.07:1, about 1.08:1, about 1.09:1, about 1.1:1, about 1.11:1, about 1.12:1, about 1.13:1, about 1.14:1, or about 1.15:1, or any ratio within a range defined by any two of the aforementioned ratios, for example, 1.01:1 to 1.15:1, 1.01:1 to 1.1:1, 1.05:1 to 1.1:1, or 1.1:1 to 1.15:1. In some embodiments, the mass ratio of the cationic polymer and the capping molecules is 100:1, 100:2, 100:3, 100:4, 100:5, 100:6, 100:7, 100:8, 100:9, 100:10, 100:15, 100:20, 100:25, 100:30, 100:35, 100:40, 100:45, 100:50, 100:55, 100:60, 100:65, 100:70, 100:75, 100:80, 100:85, 100:90, 100:95, 100:100, 100:150, 100:200, 100:300, 100:400, or 100:500, or about 100:1, about 100:2, about 100:3, about 100:4, about 100:5, about 100:6, about 100:7, about 100:8, about 100:9, about 100:10, about 100:15, about 100:20, about 100:25, about 100:30, about 100:35, about 100:40, about 100:45, about 100:50, about 100:55, about 100:60, about 100:65, about 100:70, about 100:75, about 100:80, about 100:85, about 100:90, about 100:95, about 100:100, about 100:150, about 100:200, about 100:300, about 100:400, or about 100:500, or any ratio within a range defined by any two of the aforementioned ratios, for example, 100:1 to 100:500, 100:1 to 100:25, 100:10 to 100:100, or 100:100 to 100:500. In some embodiments, the capped cationic polymer is POLY1, POLY2, POLY3, POLY4, POLY5, POLY6, POLY7, or POLY8, or any combination thereof. In some embodiments, the cationic polymers and capped cationic polymers are synthesized according to the ratios and components shown in Table 2.

Some aspects of the present disclosure relate to capped cationic polymers. In some embodiments, the capped cationic polymers are the capped cationic polymers synthesized by any one of the methods described herein. In some embodiments, the capped cationic polymers further comprise a fluorescent dye. In some embodiments, the fluorescent dye is DyLight 488, DyLight 550, or DyLight 650.

Some aspects of the present disclosure relate to labeling a cell. In some embodiments, the methods comprise contacting the cell with a cationic barcode, wherein the cationic barcode comprises a cationic polymer and a nucleic acid barcode, wherein the cationic polymer permits the nucleic acid barcode to access the cytoplasm of the cell. In some embodiments, the nucleic acid is DNA or RNA. In some embodiments, the nucleic acid is single stranded DNA (ssDNA). In some embodiments, the nucleic acid has a length of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, or 5000 nucleotides in length, or any length within a range defined by any two of the aforementioned lengths, for example, 10 to 5000 nucleotides, 100 to 1000 nucleotides, 200 to 500 nucleotides, 10 to 500 nucleotides, or 400 to 5000 nucleotides in length. In some embodiments, the cationic polymer is any one of the cationic polymers described herein. In some embodiments, the cationic polymer is a cationic polymer synthesized by any one of the methods described herein. In some embodiments, the cell is part of a tissue, organoid, or spheroid, or any combination thereof. In some embodiments, the nucleic acid has the sequence of SEQ ID NO: 2-4.

Some aspects of the present disclosure relate to methods of multiplexed barcoding of a population of cells. In some embodiments, the methods comprise contacting the population of cells with one or more cationic barcodes, wherein each of the cationic barcodes comprises a cationic polymer and a nucleic acid barcode of a unique sequence and sequencing the nucleic acid barcodes of the one or more cationic barcodes by single cell RNA-seq, thereby identifying individual cells as belonging to the population of cells by the sequences of the nucleic acid barcodes of the individual cells. In some embodiments, the cationic polymer is any one of the cationic polymers described herein. In some embodiments, the cationic polymer is a cationic polymer synthesized by any one of the methods described herein. In some embodiments, the nucleic acid barcode is a ssDNA barcode and sequencing the nucleic acid barcodes comprises amplifying the ssDNA barcode. In some embodiments, the nucleic acid barcode has the sequence of SEQ ID NO: 2-4. In some embodiments, the population of cells is part of a tissue, organoid, or spheroid. In some embodiments, the population of cells is part of a liver organoid or a foregut spheroid. In some embodiments, the population of cells comprises two or more subpopulations of cells, wherein each subpopulation of cells is from a unique individual and the population of cells is formed by combining the two or more subpopulations of cells. In some embodiments, contacting the population of cells comprises contacting each of the two or more subpopulations of cells with a unique cationic barcode before the population of cells is formed by combining the two or more subpopulations of cells. In some embodiments, sequencing comprises sequencing the unique cationic barcode of each of the two or more subpopulations of cells, thereby identifying individual cells as belonging to one of the two or more subpopulations of cells by the sequences of the nucleic acid barcodes of the individual cells.

Embodiments of the present disclosure provided herein are described by way of the following numbered alternatives:

1. A method for labeling a cell, comprising the step of contacting a cell with a cationic polymer comprising a nucleotide.

2. The method of alternative 1, further comprising labeling a cell, tissue, or organoid assembly with said polymer comprising a nucleotide.

3. The method of alternative 1 or 2, wherein said cationic polymer comprising a nucleotide is terminated with a primary, secondary, tertiary amine, or quaternary ammonium cation.

4. The method of any preceding alternative, wherein said nucleotide is single or double stranded.

5. The method of any preceding alternative, wherein said nucleotide is single stranded, and wherein said polymer comprising the nucleotide is used for DNA barcoding or FISH experiments.

6. The method of any preceding alternative, wherein said nucleotide has a length of from about 50 to about 50,000 base pairs.

7. The method of any preceding alternative, wherein said nucleotide is single stranded.

8. The method of any preceding alternative, wherein said nucleotide is single stranded.

9. The method of any preceding alternative, wherein said cationic polymer integrates into a cellular component.

10. The method of any preceding alternative, wherein said cationic polymer integrates into an intracellular component.

11. The method of any preceding alternative, comprising assessing nucleotide binding by electrophoresis.

12. The method of any preceding alternative, wherein said nucleotide serves as a barcode, comprising quantifying a temporospatial distribution of said barcode within an organoid, cell, or spheroid by flow cytometry, confocal microscopy, and combinations thereof.

13. The method of any preceding alternative, wherein said nucleotide serves as a barcode, comprising amplifying said barcode, wherein said barcode comprises a tag.

14. The method of any preceding alternative, wherein said nucleotide serves as a barcode for identifying one or more cell types.

15. The method of any preceding alternative, wherein said nucleotide serves as a barcode, comprising using said barcode for identifying a donor of a cell.

16. The method of any preceding alternative, wherein said nucleotide serves as a barcode, comprising using said barcode for quantifying one or more features of a cell.

17. The method of any preceding alternative, wherein said method does not include use of an antibody.

18. A composition for labeling of a cell, comprising a cationic polymer synthesized from acrylate monomers comprising at least two acrylate functional groups and a terminal small amine-containing molecule.

19. The composition of alternative 18, wherein said cationic polymer is a branched polymer.

20. The composition of alternative 18 or 19, wherein said composition comprises a biological buffer, preferably a 10 mM to 25 mM biological buffer, preferably having a pH of about 7.4

21. The composition of alternative 20, wherein said biological buffer is HEPES.

22. The method of any of alternatives 1 to 17, wherein said method is carried out at a pH of from about 7 to about 8.

23. A method for making a polymer-nucleotide barcode, comprising:

diluting a nucleotide (“DNA barcode”) at a concentration between about 1 μg to about 25 μL in a buffer to form a nucleotide solution;

providing a polymer according to any preceding alternative in an equal volume of buffer using in said diluting stem to form a polymer solution; and

mixing said nucleotide solution with said polymer solution.

### BRIEF DESCRIPTION OF THE DRAWINGS

In addition to the features described above, additional features and variations will be readily apparent from the following descriptions of the drawings and exemplary embodiments. It is to be understood that these drawings depict embodiments and are not intended to be limiting in scope.

FIG. 1A depicts an embodiment of the synthesis and barcoding schematic.

FIG. 1B depicts an embodiment of the reagents used in the creation of the POLY-seq system. Three reagents are used to generate the acrylate-terminated polymer: poly(ethylene glycol) diacrylate Mₙ=250 (D8), di(trimethylolpropane) tetraacrylate (V5), and 3-amino-1-propanol (S3). Polymers are then capped with one of four reagents (C1-C4)

FIG. 1C depicts an embodiment of a ¹H NMR spectrum of acrylated-terminated (POLY-ac) and spermine capped POLY2 with resonance from terminal alkenes highlighted by the dashed box.

FIG. 1D depicts an embodiment of a viability screening of POLY-seq vectors at concentrations 0.1-100 μg/mL incubated with 72.3 iPSCs for 24 hours against control vectors Lipofectamine 3000 and Mirus TransiT. ***=p<0.001, n=3.

FIG. 1E depicts an embodiment of viability screening of POLY-seq vectors with ESH1 and 1383D6 iPSCs.

FIG. 1F depicts an embodiment of a gel electrophoresis of ssDNA barcodes bound by POLY-seq polymers at indicated mass ratios.

FIG. 2A depicts an embodiment of FACS of fused spheroids pre-tagged with DyLight 488 or DyLight 650 conjugated POLY-seq vectors demonstrating singlet and double labeling.

FIG. 2B depicts an embodiment of quantification of total labeled and double labeled cells by FACS.

FIG. 2C depicts an embodiment of FACS analysis of mixed HLOs individually tagged with DyLight conjugated POLY2.

FIG. 2D depicts an embodiment of quantification of total HLO labeling by FACS analysis of FIG. 2C.

FIG. 2E depicts an embodiment of confocal immunofluorescence micrographs of lysosomes, POLY-seq vectors, mitochondria, and F-actin used to track localization of vectors within HLOs three hours post tagging. Whole HLOs are shown with POLY-seq fluorescence and F-actin staining. Scale bar=50 μm. Inset images show lysosomal colocalization. Scale bar=10 μm.

FIG. 2F depicts an embodiment of confocal imaging of POLY-seq labeled anterior foregut (upper portion, brighter) and posterior foregut (lower portion, dimmer) fused spheroids.

FIG. 3A depicts an embodiment of UMAP analysis of barcode expression in three individually tagged HLO samples.

FIG. 3B depicts an embodiment of graphs showing percentage of cells aligned to each of the three barcodes within each sample with inset targeting accuracy (94%).

FIG. 3C depicts an embodiment of high sensitivity UMAP clustering showing (i) all clustered cells and (ii) only clustered cells containing barcode reads from POLY-seq tagging. Targeting by cluster and percent coverage across all clusters is shown for sample E2. Also depicted is an embodiment of UMAP analysis and clustering of sample E3 showing (i) all cells and (ii) all cells associated with barcode E3 (top) and sample E4 showing (i) all cells and (ii) all cells associated with barcode E4 (bottom).

FIG. 3D depicts an embodiment of hashing analysis performed in Seurat for identification of doublet, negative, and singlet labeled cells for samples E2, E3, and E4 and as an average across all samples.

FIG. 3E depicts an embodiment of the number of unique detected genes (UMI) and total RNA per cell, and gene expression amongst integrated negative and single-labeled cells.

FIG. 4A depicts an embodiment of HLO hepatic lineages identified by gene expression and respective barcoded populations contained within each expressed population for: hepatocytes (HNF4α, ASGR1, CEBPA, RBP4), stellate cells (COL1A2, SPARC, TAGLN), and biliary cells (KRT7, TACSTD2, SPP1).

FIG. 4B depicts an embodiment of barcode expression within biliary, hepatocyte, and stellate populations for samples E2, E3, and E4.

FIG. 4C depicts an embodiment of heatmaps and UMAP clustering of singlet-barcoded sub-populations split by number of uniquely detected genes (High UMI >1350) and (Low UMI <1350) showing barcode representation across clusters in both sub-populations.

### DETAILED DESCRIPTION

Disclosed herein are embodiments of a polymer-based molecular barcode labeling system (termed “POLY-seq”), synthesized with low cost, commercially available reagents capable of binding standard hashing oligonucleotides (“oligos”) in 10 minutes. The POLY-seq system successfully labels cells within a cell population. In some embodiments, the cell population is an anterior foregut spheroid population, a posterior foregut spheroid population, or a human liver organoid population. This system achieves functional barcoding within one hour using standard hashing oligos, in some embodiments allowing for the correct identification of barcode labels in 90% of cells derived from human liver organoids prepared on the 10× Genomics single-cell RNA-seq platform, providing an opportunity for pooled heterogeneous sample multiplexing in a rapid, cost-efficient manner.

Next-generation sequencing (NGS) provides a powerful tool for unparalleled investigative depth into transcriptomic and genomic profiles. Single-cell techniques offer the ability for high-resolution analysis of a heterogeneous sample. However, with the caveat of only one experimental condition per library preparation, elevating the costs to run multiple samples as the preparation of multiple libraries is required. For example, single-cell RNA sequencing (scRNA-seq) uses a dual barcoding scheme such that every RNA strand captured for sequencing receives its own strand-specific barcode while all RNA strands captured for a single cell receive their own cell-specific barcode. As larger sequencers possess the capacity to run multiple single-cell experiments in parallel with adequate sequencing depth, scRNA-seq preparation generally affixes a third experiment-specific index barcode such that multiple experiments may be pooled and run in parallel. This multiplexing allows for enhanced throughput and reduced cost per number of reads. However, as affixing the index is performed during the final steps of library preparation, samples must be prepared individually to receive distinct indices, potentially generating high costs when adequate read depth allows for separate samples to be pooled together. This sample pooling prior to single-cell processing necessitates a methodology capable of heterogeneously tagging samples with barcodes readable by NGS platforms.

One common technique for cell labeling employs barcode-conjugated antibodies. This method takes advantage of specific labeling offered by antibodies to not only differentiate targets but allows for expression quantification. Through innate barcoding heterogeneity derived from the specific labeling of multiple samples, this further allows sample multiplexing and super-loading. A complementary technology employs modification of fatty acids for non-selective integration into cell membranes. This method seeks to enhance targeting ubiquity at the expense of specificity juxtaposed with antibody labeling. While antibody-based barcoding methods allow for quantification of cell surface protein expression or specific subpopulation tagging and lipid methods allow for more universal barcode integration, their preparation can be costly or time consuming in the creation of custom libraries. Barcodes are directly, covalently conjugated to the labeling mediators, reducing flexibility especially in the case where custom sample barcoding is useful for labeling a heterogeneous population for multiplex applications. Other techniques rely upon genetic diversity to drive demultiplexing through bioinformatic processing or the expression of barcoding sequences from the creation and generation of viral libraries. While viral methods are convenient for long term lineage tracing, the generation and application of viral libraries with high transduction efficiency for sufficient barcode representation in multiplex applications may be restrictive for short-term labeling. Therefore, there exists an opportunity for the development of a fast, efficient, ubiquitous sample-specific barcoding tool allowing for the creation of custom barcoding pools requiring minimal preparation, significantly enhancing throughput and reducing sequencing cost through multiplexing juxtaposed with the current common sample preparation strategy of one sample per experiment.

Polymer-based transfection techniques have previously been investigated for their ability to deliver an array of functional DNA and/or RNA encoding a sequence of choice or for modification of protein expression. Operating on the general principle of ionic interaction, polymer vectors employing charge-based methodology rely upon cationic charge of the polymer to bind DNA/RNA through interaction with the anionic charges populating the backbone of nucleic acids and to interact with cell surfaces. It is upon this principle that allow for the direct translation of polymers from transfection mediators to barcoding vectors with previous applications focused on tracking delivery and distribution of information in vivo. However, optimization of formulations for efficient single cell multiplexing applications has yet to be fully explored. The two defining characteristics of a system for barcoding with applicability to sample multiplexing are universal binding regardless of sample heterogeneity and, importantly, binding fidelity. When utilizing sample multiplexing, a particular cell, no matter how clearly the transcriptome or genome is sequenced, must possess a defined, sample-specific barcode identifiable in downstream bioinformatics processing. In a heterogeneous sample, universal labeling serves to deliver an unbiased method with which samples may be pooled. Binding fidelity ensures that once cells are tagged with a sample-specific barcode, barcoding vectors will remain bound to original cells during multiplexing and will not migrate to other cells that otherwise would lower the confidence at which a sequenced cell may be assigned to a specific sample. These two parameters used as quantification metrics during the development of POLY-seq vectors as described herein.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, 20%, 30%, 40%, 50%, 60%, 70%, 80%, 90%, 95%, 98%, 99/c, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Cationic Polymers and Methods of Making

The term “cationic polymer” as used herein has its plain and ordinary meaning as understood in light of the specification and refers to high molecular weight polymeric compounds that exhibit positive (cationic) charges on its surface. In some embodiments, the positive charges are due to amine groups on the cationic polymer. The cationic polymer may be a linear polymer, branched polymer, randomly branched polymer, dendrimer, block polymer, or graft polymer. In some embodiments, these different polymeric structures alter the properties of the cationic polymer. For the purposes of delivery into cells, cationic polymers can bind to the negatively charged phosphate backbone of nucleic acids (e.g. DNA or RNA) to form a polymer/nucleic acid complex. The cationic polymer may also alter the three-dimensional structure of the nucleic acid, for example, compacting the nucleic acid or making it less accessible to nucleases. Cationic polymers are also selected according to qualities such as number or density of cationic charges or regions, safety, toxicity, biodegradability, ease of use, ease of synthesis, efficiency in nucleic acid complex formation, efficiency in nucleic acid delivery, aggregation tendency, ability for additional modifications with functional groups, or cost, or any combination thereof. While still not fully understood, cationic polymers deliver complexed nucleic acids to cells by interacting with the cell's plasma membrane through charge interactions, internalization into the cell by endocytosis, and release of the nucleic acid into the cell cytoplasm. In the case of nucleic acid payloads that are intended for gene expression, these nucleic acids can either be translated directly by ribosomes (as is the case with RNA) or translocate to the nucleus to be transcribed as episomes (as DNA). For barcoding applications, the nucleic acid payloads can be analyzed, such as by sequencing, at any step of this process. Examples of cationic polymers known in the art include but are not limited to polyethylenimine (PEI), poly-L-lysine (PLL), chitosan, DEAE-dextran, or polyamidoamine (PAMAM). Some cationic polymers can be combined with lipid-based transfection reagents to enhance delivery into cells. Examples of commercial transfection reagents, which may or may not comprise cationic polymers, include but are not limited to Lipofectamine, TransIT, or Fugene.

Described herein are methods of synthesizing a cationic polymer. In some embodiments, the methods comprise using diacrylate monomers and alkanolamines. In some embodiments, the acrylate functional group of the diacrylate monomers and the amine functional group of the alkanolamines react according to a Michael addition reaction to form an acrylate-amino adduct. In some embodiments, the Michael addition is an aza-Michael addition. In some embodiments, the methods comprise reacting a plurality of diacrylate monomers and a plurality of alkanolamines results in a diacrylate/alkanolamine polymer. In some embodiments, the diacrylate monomer is a poly(ethylene glycol) diacrylate (“D8”) monomer or a di(trimethylolpropane) tetraacrylate (“V5”) monomer, or both. In some embodiments, the diacrylate monomer is a linear diacrylate monomer. In some embodiments, the diacrylate monomer has the structure

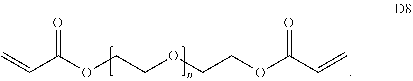

In some embodiments, the diacrylate monomer is a branched diacrylate monomer. In some embodiments, the diacrylate monomer has the structure

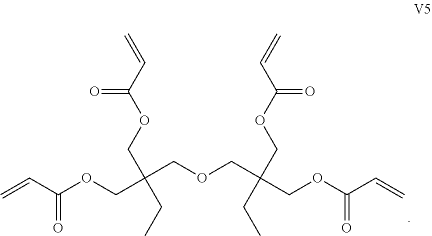 [SMILES: C=CC(=O)OCC(CC)(COCC(CC)(COC(=O)C=C)COC(=O)C=C)COC(=O)C=C]

In some embodiments, the poly(ethylene glycol) diacrylate is poly(ethylene glycol) diacrylate Mₙ=250. In some embodiments, the alkanolamine is 3-amino-1-propanol (“S3”). In some embodiments, the alkanolamine has the structure

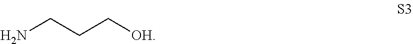

In some embodiments, the methods comprise reacting D8 monomers with S3 monomers, resulting in a D8/S3 polymer. In some embodiments, the methods comprise contacting D8 and S3, resulting in a D8/S3 polymer. In some embodiments, the D8 and S3 are reacted by Michael Addition. In some embodiments, the D8/S3 polymer is produced by Michael Addition by contacting D8 and S3. In some embodiments, the D8/S3 polymer is a linear polymer. In some embodiments, the D8/S3 polymer comprises one or two acrylate groups. In some embodiments, the D8/S3 polymer is a cationic polymer. In some embodiments, the amount of D8 is greater than the amount of S3. In some embodiments, D8 is more abundant than S3. In some embodiments, D8 is in excess. In some embodiments, the molar ratio of D8 to S3 is greater than 1. In some embodiments, the molar ratio of D8 to S3 is, is about, is at least, is at least about, is not more than, or is not more than about, 1.01:1, 1.02:1, 1.03:1, 1.04:1, 1.05:1, 1.06:1, 1.07:1, 1.08:1, 1.09:1, 1.1:1, 1.11:1, 1.12:1, 1.13:1, 1.14:1, or 1.15:1, or any ratio within a range defined by any two of the aforementioned ratios, for example, 1.01:1 to 1.15:1, 1.01:1 to 1.1:1, 1.05:1 to 1.1:1, or 1.1:1 to 1.15:1. In some embodiments, the molar ratio of D8 to S3 is, is about, is at least, is at least about, is not more than, or is not more than about, 1.05:1. In some embodiments, the molar ratio of D8 to S3 is, is about, is at least, is at least about, is not more than, or is not more than about, 1.1:1. In some embodiments, the methods comprise reacting a mixture of D8 monomers and V5 monomers with S3 monomers, resulting in a D8/V5/S3 polymer. In some embodiments, the methods comprise contacting D8, V5, and S3, resulting in a D8/V5/S3 polymer. In some embodiments, the D8/V5/S3 polymer is a cationic polymer. In some embodiments, the D8/V5/S3 polymer is a branched polymer. In some embodiments, the D8/V5/S3 polymer comprises more than two terminal acrylate groups. In some embodiments, the amount of D8 and V5 is greater than the amount of S3. In some embodiments, D8 and V5 is more abundant than S3. In some embodiments, D8 and V5 are in excess. In some embodiments, the molar ratio of D8 to S3 is greater than 1. In some embodiments, the molar ratio of D8 to S3 is, is about, is at least, is at least about, is not more than, or is not more than about, 1.01:1, 1.02:1, 1.03:1, 1.04:1, 1.05:1, 1.06:1, 1.07:1, 1.08:1, 1.09:1, 1.1:1, 1.11:1, 1.12:1, 1.13:1, 1.14:1, or 1.15:1, or any ratio within a range defined by any two of the aforementioned ratios, for example, 1.01:1 to 1.15:1, 1.01:1 to 1.1:1, 1.05:1 to 1.1:1, or 1.1:1 to 1.15:1. In some embodiments, the molar ratio of D8 to S3 is, is about, is at least, is at least about, is not more than, or is not more than about, 1.05:1. In some embodiments, the molar ratio of D8 to S3 is, is about, is at least, is at least about, is not more than, or is not more than about, 1.1:1. In some embodiments, the molar ratio of V5 to S3 is less than 1. In some embodiments, the molar ratio of V5 to S3 is, is about, is at least, is at least about, is not more than, or is not more than about, 0.1:1, 0.2:1, 0.3:1, 0.4:1, 0.5:1, 0.6:1, 0.7:1, 0.8:1, 0.9:1, or 1:1, or any ratio within a range defined by any two of the aforementioned ratios, for example, 0.1:1 to 1:1, 0.5:1 to 0.8:1, 0.1:1 to 0.5:1, or 0.5:1 to 1:1. In some embodiments, the molar ratio of D8 to V5 is greater than 1. In some embodiments, the molar ratio of D8 to V5 is, is about, is at least, is at least about, is not more than, or is not more than about, 1.1:1, 1.2:1, 1.3:1, 1.4:1, 1.5:1, 1.6:1, 1.7:1, 1.8:1, 1.9:1, or 2.0:1, or any ratio within a range defined by any two of the aforementioned ratios, for example, 1.1:1 to 2.0:1, 1.3:1 to 1.8:1, 1.1:1 to 1.5:1, or 1.5:1 to 2.0:1. In some embodiments, the molar ratios of D8, V5, and S3 are provided in Table 2.

In some embodiments, the cationic polymer synthesized by any one of the methods described herein are acrylate terminated, wherein the cationic polymer comprises one or more acrylate functional groups. In some embodiments, the one or more acrylate functional groups are further reacted. In some embodiments, the cationic polymer is reacted with one or more capping molecules to form a capped cationic polymer. In some embodiments, the cationic polymer is contacted with one or more capping molecules to form a capped cationic polymer. In some embodiments, the one or more capping molecules comprise amine groups. In some embodiments, the amine groups of the one or more capping molecules reacts with the one or more acrylate function groups by Michael addition. In some embodiments, the Michael addition is an aza-Michael addition. In some embodiments, the capping molecule is one or more (e.g. at least 1, 2, 3, 4) of 1,4-bis(3-aminopropyl)piperazine (“C1”), spermine (“C2”), polyethylenimine (“C3”), or 2,2-dimethyl-1,3-propanediamine (“C4”), or any combination thereof. In some embodiments, the capping molecule has the structure

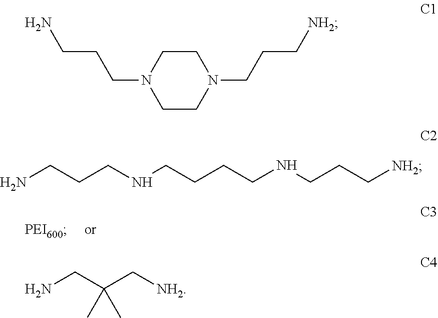

In some embodiments, the cationic polymer and the capping molecule are contacted at a certain mass ratio. In some embodiments, the cationic polymer and the capping molecule are contacted at a mass ratio that is greater than 1. In some embodiments, the cationic polymer and the capping molecule are contacted at a mass ratio that is less than 1. In some embodiments, the cationic polymer and the capping molecule are contacted at a mass ratio that is, is about, is at least, is at least about, is not more than, or is not more than about, 100:1, 100:2, 100:3, 100:4, 100:5, 100:6, 100:7, 100:8, 100:9, 100:10, 100:15, 100:20, 100:25, 100:30, 100:35, 100:40, 100:45, 100:50, 100:55, 100:60, 100:65, 100:70, 100:75, 100:80, 100:85, 100:90, 100:95, 100:100, 100:150, 100:200, 100:300, 100:400, or 100:500, or any ratio within a range defined by any two of the aforementioned ratios, for example, 100:1 to 100:500, 100:1 to 100:25, 100:1 to 100:100, 100:10 to 100:100, or 100:100 to 100:500. In some embodiments, the cationic polymer and the capping molecule are contacted at a mass ratio provided in Table 2. In some embodiments, the capped cationic polymer does not comprise any acrylate groups. In some embodiments, the capped cationic polymer is one or more (e.g. 1, 2, 3, 4, 5, 6, 7, 8) of vectors POLY1, POLY2, POLY3, POLY4, POLY5, POLY6, POLY7, or POLY8, or any combination thereof. In some embodiments, the capped cationic polymer is vector POLY1. In some embodiments, the capped cationic polymer is vector POLY2. In some embodiments, the capped cationic polymer is vector POLY3. In some embodiments, the capped cationic polymer is vector POLY4. In some embodiments, the capped cationic polymer is the vector POLY5. In some embodiments, the capped cationic polymer is vector POLY6. In some embodiments, the capped cationic polymer is vector POLY7. In some embodiments, the capped cationic polymer is vector POLY8. In some embodiments, the capped cationic polymer is any one of the capped cationic polymers provided in Table 2. In some embodiments, the capped cationic polymer is a capped cationic polymer synthesized according to the molar ratios or mass ratios provided in Table 2.

In some embodiments, the cationic polymer is synthesized by mixing a diacrylate monomer disclosed herein and an amino alcohol (alkanolamine) disclosed herein to form an uncapped acrylate terminated cationic polymer. In some embodiments, the diacrylate monomer and amino alcohol are reacted at a temperature that is, is about, is at least, is at least about, is not more than, or is not more than about, 10° C., 20° C., 30° C., 40° C., 50° C., 60° C., 70° C., 80° C., 85° C., 86° C., 87° C., 88° C., 89° C., 90° C., 91° C., 92° C., 93° C., 94° C., 95° C., 96° C., 97° C., 98° C., 99° C., or 100° C., or any temperature within a range defined by any two of the aforementioned temperatures, for example, 10° C. to 100° C., 60° C. to 95° C., 85° C. to 99° C., 10° C. to 90° C., or 85° C. to 100° C. In some embodiments, the diacrylate monomer and amino alcohol are reacted at a temperature that is, is about, is at least, is at least about, is not more than, or is not more than about, 90° C. In some embodiments, the diacrylate monomer and amino alcohol are reacted for a number of hours that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or any number of hours within a range defined by any two of the aforementioned number of hours, for example, 1 to 48 hours, 10 to 30 hours, 20 to 25 hours, 1 to 24 hours, or 24 to 48 hours. In some embodiments, the diacrylate monomer and amino alcohol are reacted for a number of hours that is, is about, is at least, is at least about, is not more than, or is not more than about, 24 hours.

In some embodiments, the uncapped acrylate terminated cationic polymer is capped, forming a capped cationic polymer, by the addition of a capping molecule, wherein the capping molecule is a molecule comprising a primary or secondary amine. In some embodiments, the uncapped acrylate terminated cationic polymer is reacted with the capping molecule at a temperature that is, is about, is at least, is at least about, is not more than, or is not more than about, 10° C., 20° C., 30° C., 40° C., 50° C., 60° C., 70° C., 80° C., 85° C., 86° C., 87° C., 88° C., 89° C., 90° C., 91° C., 92° C., 93° C., 94° C., 95° C., 96° C., 97° C., 98° C., 99° C., or 100° C., or any temperature within a range defined by any two of the aforementioned temperatures, for example, 10° C. to 100° C., 60° C. to 95° C., 85° C. to 99° C., 10° C. to 90° C., or 85° C. to 100° C. In some embodiments, the uncapped acrylate terminated cationic polymer is reacted with the capping molecule at a temperature that is, is about, is at least, is at least about, is not more than, or is not more than about, 50° C. In some embodiments, the uncapped acrylate terminated cationic polymer is reacted with the capping molecule at a temperature that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or any number of hours within a range defined by any two of the aforementioned number of hours, for example, 1 to 48 hours, 10 to 30 hours, 20 to 25 hours, 1 to 24 hours, or 24 to 48 hours. In some embodiments, the uncapped acrylate terminated cationic polymer is reacted with the capping molecule at a temperature that is, is about, is at least, is at least about, is not more than, or is not more than about, 24 hours. In some embodiments, the capped cationic polymers are stored at a temperature is, is about, is at least, is at least about, is not more than, or is not more than about, −20° C.

In some embodiments, the cationic polymers or capped cationic polymers are conjugated with a fluorescent tag. In some embodiments, the cationic polymers or capped cationic polymers are conjugated with a fluorescent tag using amine-reactive conjugation. In some embodiments, the cationic polymers or capped cationic polymers are conjugated using N-hydroxysuccinimide ester conjugation. In some embodiments, the fluorescent tag comprises an N-hydroxysuccinimide ester functional group. In some embodiments, the fluorescent tag is DyLight 488, DyLight 550, or DyLight 650.

Described herein are cationic polymers, capped cationic polymers, or both, or compositions thereof. In some embodiments, the cationic polymer is the cationic polymer produced by any one of the methods described herein. In some embodiments, the capped cationic polymer is the capped cationic polymer produced by any one of the methods described herein. In some embodiments, the capped cationic polymer is one or more (e.g. 1, 2, 3, 4, 5, 6, 7, 8) of vectors POLY1, POLY2, POLY3, POLY4, POLY5, POLY6, POLY7, or POLY8, or any combination thereof. In some embodiments, the capped cationic polymer is vector POLY1. In some embodiments, the capped cationic polymer is vector POLY2. In some embodiments, the capped cationic polymer is vector POLY3. In some embodiments, the capped cationic polymer is vector POLY4. In some embodiments, the capped cationic polymer is the vector POLY5. In some embodiments, the capped cationic polymer is vector POLY6. In some embodiments, the capped cationic polymer is vector POLY7. In some embodiments, the capped cationic polymer is vector POLY8. In some embodiments, the capped cationic polymer is any one of the capped cationic polymers provided in Table 2. In some embodiments, the capped cationic polymer is a capped cationic polymer synthesized according to the molar ratios or mass ratios provided in Table 2. In some embodiments, the cationic polymer or capped cationic polymer, or both, further comprise a fluorescent dye. In some embodiments, the fluorescent dye is DyLight 488, DyLight 550, or DyLight 650, or any combination thereof.

The terms “barcode” and “barcoding” have their plain and ordinary meaning as understood in light of the specification and refer to the use of short nucleic acids with known sequences in order to label cells or a component of cells (e.g. genomic DNA, RNA, mRNA, miRNA, siRNA, proteins, peptides, polypeptides) and identify the cells or component of cells by sequencing. In some embodiments, the nucleic acids are double stranded DNA (dsRNA), single stranded DNA (ssDNA), double stranded RNA (dsRNA), or single stranded RNA (ssRNA). The nucleic acids comprise a unique barcode sequence as well as one or more constant adapter sequences that is the same among different nucleic acid barcodes. Typically, the one or more constant adapter sequences are at opposite ends of the nucleic acid strand (i.e. at the 5′ and 3′ end) and are flanking the unique barcode sequence. These one or more constant adapter sequences are used as primer annealing regions so that the same primers can be used for the entire set of different barcodes. Amplifying the barcodes with the primers will result in amplification of the unique barcode sequence, which is necessary to be able to detect the unique barcode sequences using current methods. The nucleic acid barcodes may be modified or conjugated in some way, such as with an antibody, to be able to bind to different components of the cell. For cell barcoding applications, one cell can be differentiated from another cell within a population or mixture of cells based on the amplified sequences of the unique barcodes in each of the cells. As used herein, cationic polymers are used to deliver the nucleic acid barcodes into the cells within a population of cells. Analysis of the population of cells by single cell sequencing techniques such as single cell RNA sequencing (scRNA-seq) while the cells have these barcodes permit identification of individual cells and their constituent transcriptomic profile. In some embodiments, the population of cells is comprised of two or more subpopulations of cells. By delivering different and unique barcodes to each of the two or more subpopulations of cells, sequencing the barcodes permits identification of a cell as belonging to one of the two or more subpopulations of cells even if the two or more subpopulations are mixed together in a sample.

In some embodiments, the cationic polymer and nucleic acid barcode are combined in solution to form a cationic barcode. In some embodiments, the cationic polymer and nucleic acid barcode are combined in a w/w ratio that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 w/w ratio cationic polymer:nucleic acid barcode, or any w/w ratio within a range defined by any two of the aforementioned w/w ratios, for example, 1 to 80, 10 to 60, 20 to 50, 1 to 60, or 10 to 80 w/w ratio. In some embodiments, the cationic polymer and nucleic acid barcode are combined at a 2 w/w ratio. In some embodiments, the cationic polymer and nucleic acid barcode are combined at a 5 w/w ratio. In some embodiments, the cationic polymer and nucleic acid barcode are combined at a 10 w/w ratio. In some embodiments, the cationic polymer and nucleic acid barcode are combined at a 20 w/w ratio. In some embodiments, the cationic polymer and nucleic acid barcode are combined at a 40 w/w ratio. In some embodiments, the cationic polymer and nucleic acid barcode are combined at a 60 w/w ratio. In some embodiments, the cationic polymer and nucleic acid barcode are combined in an aqueous solution. In some embodiments, the cationic polymer and nucleic acid barcode are combined in growth medium. In some embodiments, the cationic polymer and nucleic acid barcode are combined in mTeSR medium.

Described herein are methods of labeling or barcoding a cell. In some embodiments, some embodiments, the methods comprise contacting the cell with a cationic barcode. In some embodiments, the cationic barcode comprises a cationic polymer and a nucleic acid barcode. In some embodiments, the cationic polymer permits the nucleic acid barcode to access the cytoplasm of the cell. In some embodiments, the nucleic acid barcode is the nucleic acid barcode described herein and elsewhere. In some embodiments, the nucleic acid is DNA or RNA, or both. In some embodiments, the nucleic acid is ssDNA. In some embodiments, the nucleic acid has a length that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, or 5000 nucleotides in length, or any length within a range defined by any two of the aforementioned lengths, for example, 10 to 5000 nucleotides, 100 to 1000 nucleotides, 200 to 500 nucleotides, 10 to 500 nucleotides, or 400 to 5000 nucleotides in length. In some embodiments, the nucleic acid has the sequence of SEQ ID NO: 2-4. In some embodiments, the cationic polymer is the cationic polymer produced by any one of the methods described herein. In some embodiments, the cationic polymer is the capped cationic polymer produced by any one of the methods described herein. In some embodiments, the cell is within a population of cells. In some embodiments, the cell is part of a tissue, organoid, or spheroid, or any combination thereof. In some embodiments, the cell is part of a liver organoid or a foregut spheroid. In some embodiments, the cell is part of a liver organoid. In some embodiments, the cell is contacted with the cationic barcode for a number of hours that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or any number of hours within a range defined by any two of the aforementioned number of hours, for example, 1 to 48 hours, 10 to 30 hours, 20 to 25 hours, 1 to 24 hours, or 24 to 48 hours. In some embodiments, the methods comprise sequencing the cationic barcode. In some embodiments, the methods comprise sequencing the cationic barcode by single cell sequencing. In some embodiments, the methods comprise sequencing the cationic barcode by scRNA-seq.

Disclosed herein are methods of multiplexed barcoding of a population of cells. As discussed herein and elsewhere, it is advantageous to multiplex sequencing technologies using barcodes in order to increase throughput of data acquisition (e.g. running multiple samples within each run of sequencing). In some embodiments, the methods comprise contacting the population of cells with one or more cationic barcodes. In some embodiments, the one or more cationic barcodes each comprise a cationic polymer and a nucleic acid barcode of a unique sequence. In some embodiments, the cationic polymer is any cationic polymer described herein, or the cationic polymer synthesized by any one of the methods described herein. In some embodiments, the cationic polymer is any capped cationic polymer described herein, or the capped cationic polymer synthesized by any one of the methods described herein. In some embodiments, the cationic polymer is one or more (e.g at least 1, 2, 3, 4, 5, 6, 7, 8) of vectors POLY1, POLY2, POLY3, POLY4, POLY5, POLY6, POLY7, or POLY8, or any combination thereof, as disclosed herein. In some embodiments, the nucleic acid barcode is a DNA or RNA strand. In some embodiments, the nucleic acid barcode is single stranded DNA (ssDNA). In some embodiments, the nucleic acid barcode is a ssDNA barcode. In some embodiments, the nucleic acid barcode is part of a barcoding array known in the art. In some embodiments, the nucleic acid barcode is based off of the CITE-seq hashing oligomer array. In some embodiments, the nucleic acid barcode has the sequence of SEQ ID NO: 2-4. In some embodiments, the nucleic acid barcode is chemically synthesized. In some embodiments, the nucleic acid barcode comprises one or more nucleic acid modifications as described herein. In some embodiments, after contacting the population of cells with one or more cationic barcodes, the methods comprise sequencing the nucleic acid barcodes of the one or more cationic barcodes. In some embodiments, sequencing of the nucleic acid barcodes is by single cell RNA-seq (scRNA-seq). In some embodiments, the sequencing of the nucleic acid barcodes identifies individual cells as belonging to the population of cells. In some embodiments, the individual cells are identified as belonging to the population of cells by the sequences of the nucleic acid barcodes of the individual cells. In some embodiments, sequencing of the nucleic acid barcodes comprises amplifying the nucleic acid barcodes. In some embodiments where the nucleic acid barcodes are ssDNA barcodes, sequencing the nucleic acid barcodes comprises amplifying the ssDNA barcodes.

In some embodiments, the capped cationic polymer and nucleic acid barcode are combined at a w/w capped cationic polymer:nucleic acid barcode ratio that is, is about, is at least, is at least about, is not more than, or is not more than about, 1/1, 2/1, 3/1, 4/1, 5/1, 6/1, 7/1, 8/1, 9/1, 10/1, 11/1, 12/1, 13/1, 14/1, 15/1, 16/1, 17/1, 18/1, 19/1, 20/1, 21/1, 22/1, 23/1, 24/1, 25/1, 26/1, 27/1, 28/1, 29/1 or 30/1 μg/μg, or any ratio within a range defined by any two of the aforementioned ratios, for example, 1/1 to 30/1, 10/1 to 25/1, 15/1 to 20/1, 1/1 to 20/1, or 15/1 to 30/1 w/w capped cationic polymer:nucleic acid barcode ratio. In some embodiments, for a population of cells, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg of capped cationic polymer is used, or any mass within a range defined by any two of the aforementioned masses, for example, 1 to 50 μg, 10 to 40 μg, 20 to 30 μg, 1 to 30 μg, or 20 to 50 μg. In some embodiments, the capped cationic polymer and nucleic acid barcode are combined in growth medium. In some embodiments, the growth medium is HCM. In some embodiments, the capped cationic polymer and nucleic acid barcode are allowed to complex over an amount of time that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 minutes, or any time within a range defined by any two of the aforementioned times, for example, 1 to 30 minutes, 10 to 25 minutes, 15 to 20 minutes, 1 to 20 minutes, or 10 to 30 minutes. In some embodiments, the complexed capped cationic polymer and nucleic acid barcode are contacted with a population of cells. In some embodiments, the population of cells is a liver organoid. In some embodiments, the complexed capped cationic polymer and nucleic acid barcode are contacted with the population of cells for an amount of time that is, is about, is at least, is at least about, is not more than, or is not more than about, 10, 20, 30, 40, 50, 60, 70, 80, 90, 100, 110, or 120 hours, or any time within a range defined by any two of the aforementioned times, for example, 10 to 120 hours, 30 to 100 hours, 20 to 50 hours, 10 to 30 hours, or 50 to 120 hours. In some embodiments, cellular association of the complexed capped cationic polymer and nucleic acid occurs before an amount of time that is, is about, is at least, is at least about, is not more than, or is not more than about, 1, 2, 3, 4, 5, 6, 7, 8, 9, 10, 11, or 12 hours after contacting, or any amount of time within a range defined by any two of the aforementioned times, for example, 1 to 12 hours, 2 to 10 hours, 2 to 4 hours, or 1 to 5 hours. In some embodiments, the complexed capped cationic polymer and nucleic acid colocalizes with the cellular lysosomes. In some embodiments, the population of cells is dissociated into a single cell suspension. In some embodiments, the single cell suspension is sequenced by single cell sequencing. In some embodiments, the single cell suspension is sequenced by scRNA-seq.

In some embodiments, barcoding a population of cells with a capped cationic polymer as described herein results in labeling of is, is about, is at least, is at least about, is not more than, or is not more than about, 50%, 60%, 70%, 80%, 85%, 90%, 91%, 92%, 93%, 94%, 95%, 96%, 97%, 98%, 99%, or 100% labeling of cells, or any percentage within a range defined by any two of the aforementioned percentages, for example, 50% to 100%, 80 to 95%, 85% to 94%, 50% to 90%, or 80% to 100%. In some embodiments, the sequencing is, is about, is at least, is at least about, is not more than, or is not more than about, 50%, 60%, 70%, 80%, 85%, 90%, 91%, 92%, 93%, 94%, 95%, 96%, 97%, 98%, 99%, or 100% accurate, or any percentage within a range defined by any two of the aforementioned percentages, for example, 50% to 100%, 80 to 95%, 85% to 94%, 50% to 90%, or 80% to 100%.

In some embodiments, a population of cells is prepared, obtained, or derived from more than one individual. In some embodiments, this population of cells is a “pooled population”. In some embodiments, the population of cells is prepared, obtained, or derived from a number of individuals that is, is about, is at least, is at least about, is not more than, or is not more than about, 1, 2, 3, 4, 5, 6, 7, 8, 9, 10, 20, 30, 40, 50, 60, 70, 80, 90, 100, 200, 300, 400, 500, 600, 700, 800, 900, or 1000 individuals, or any number of individuals within a range defined by any two of the aforementioned numbers, for example 1 to 1000 individuals, 10 to 500 individuals, 50 to 100 individuals, 1 to 200 individuals, or 50 to 1000 individuals. In some embodiments, the population of cells is derived from iPSCs from more than one individual. In some embodiments, the population of cells is derived from iPSCs by synchronizing the iPSCs from the more than one individual with a synchronization condition to obtain synchronized iPSCs. In some embodiments, the iPSCs are differentiated after synchronization. In some embodiments, the iPSCs are differentiated into definitive endoderm, foregut spheroid, an organoid, or a liver organoid, or any combination thereof, after synchronization. In some embodiments, the population of cells is part of a tissue, organoid, or spheroid, or any combination thereof. In some embodiments, the population of cells is a tissue, organoid, or spheroid, or any combination thereof. In some embodiments, the population of cells is part of an organoid or a foregut spheroid, or both. In some embodiments, the population of cells is an organoid or a foregut spheroid, or both. In some embodiments, the population of cells is part of a liver organoid or is a liver organoid.

In some embodiments, the population of cells from more than one individual is an organoid (“pooled organoid”). In some embodiments, the pooled organoid is prepared, obtained, or derived from a number of individuals that is, is about, is at least, is at least about, is not more than, or is not more than about, 1, 2, 3, 4, 5, 6, 7, 8, 9, 10, 20, 30, 40, 50, 60, 70, 80, 90, 100, 200, 300, 400, 500, 600, 700, 800, 900, or 1000 individuals, or any number of individuals within a range defined by any two of the aforementioned numbers, for example 1 to 1000 individuals, 10 to 500 individuals, 50 to 100 individuals, 1 to 200 individuals, or 50 to 1000 individuals. In some embodiments, the population of cells from more than one individual is an organoid derived from iPSCs from more than one individual. In some embodiments, the organoid is derived from iPSCs by synchronizing the iPSCs from the more than one individual with a synchronization condition to obtain a synchronized organoid. In some embodiments, the organoid is a liver organoid, gastric organoid, intestinal organoid, brain organoid, pulmonary organoid, esophageal organoid, bone organoid, cartilage organoid, bladder organoid, blood vessel organoid, endocrine organoid, or sensory organoid, or any combination thereof. Pooled organoids and methods of making and use thereof is explored in PCT Publication WO 2018/191673, which is incorporated herein by reference in its entirety.

In some embodiments, the population of cells comprises two or more subpopulations of cells. In some embodiments, each of the two or more subpopulation of cells is from a unique individual. In some embodiments, the population of cells is formed by combining the two or more subpopulations of cells. In some embodiments, the two or more subpopulations comprise a number of subpopulations that is, is about, is at least, is at least about, is not more than, or is not more than about, 1, 2, 3, 4, 5, 6, 7, 8, 9, 10, 20, 30, 40, 50, 60, 70, 80, 90, 100, 200, 300, 400, 500, 600, 700, 800, 900, or 1000 subpopulations, or any number of subpopulations within a range defined by any two of the aforementioned numbers, for example 1 to 1000 subpopulations, 10 to 500 subpopulations, 50 to 100 subpopulations, 1 to 200 subpopulations, or 50 to 1000 subpopulations. In some embodiments, the two or more subpopulations are from a number of individuals that is, is about, is at least, is at least about, is not more than, or is not more than about, 2, 3, 4, 5, 6, 7, 8, 9, 10, 20, 30, 40, 50, 60, 70, 80, 90, 100, 200, 300, 400, 500, 600, 700, 800, 900, or 1000 individuals, or any number of individuals within a range defined by any two of the aforementioned numbers, for example 1 to 1000 individuals, 10 to 500 individuals, 50 to 100 individuals, 1 to 200 individuals, or 50 to 1000 individuals. In some embodiments, contacting the population of cells with one or more cationic barcodes comprises contacting the population of cells with two or more cationic barcodes. In some embodiments, contacting the population of cells with one or more cationic barcode comprises contacting the population of cells with the same number of cationic barcodes as there are number of subpopulations. In some embodiments, the population of cells are contacted with a number of cationic barcodes that is at least one more than there are number of subpopulations. In some embodiments, the population of cells are contacted with a number of cationic barcodes that is, is about, is at least, is at least about, is not more than, or is not more than about, 2, 3, 4, 5, 6, 7, 8, 9, 10, 20, 30, 40, 50, 60, 70, 80, 90, 100, 200, 300, 400, 500, 600, 700, 800, 900, or 1000 cationic barcodes, or a number of cationic barcodes within a range defined by any two of the aforementioned number of cationic barcodes, for example, 2 to 1000 cationic barcodes, 10 to 500 cationic barcodes, 50 to 100 cationic barcodes, 1 to 200 cationic barcode, or 50 to 1000 cationic barcodes. In some embodiments, the population of cells is contacted with a number of cationic barcodes that is, is about, is at least, is at least about, is not more than, or is not more than about, 1, 2, 3, 4, 5, 6, 7, 8, 9, 10, 11, 12, 13, 14, 15, 16, 17, 18, 19, or 20 more cationic barcodes than there are number of subpopulations, or any number of cationic barcodes more than there are number of subpopulations, for example, 1 to 20 more, 5 to 15 more, 10 to 12 more, 1 to 10 more, or 10 to 20 more cationic barcodes than there are subpopulations in the population of cells.

In some embodiments, the population of cells is formed by combining the two or more (e.g. at least 2, 3, 4, 5, 6, 7, 8, 9, 10 50, 100, 500, 1000) subpopulations of cells. In some embodiments, the population of cells is formed by combining the two or more subpopulations of cells when the two or more subpopulation of cells are in a single cell suspension. In some embodiments, the two or more subpopulations of cells that are combined are single cell suspensions. In some embodiments, the two or more subpopulations of cells that are combined are iPSCs. In some embodiments, the two or more subpopulations of cells that are combined are foregut spheroids. In some embodiments, the two or more subpopulations of cells that are combined are foregut spheroids that are dissociated. In some embodiments, the two or more subpopulations of cells that are combined are liver organoids. In some embodiments, the two or more subpopulations of cells that are combined are liver organoids that are dissociated. In some embodiments, the two or more subpopulations of cells are cells that are synchronized with each other. In some embodiments, each of the two or more subpopulations of cells are contacted with one or more (e.g. at least 1, 2, 3, 4, 5) cationic barcodes. In some embodiments, each of the one or more cationic barcodes are unique, both among the cationic barcodes that are contacted to the same subpopulation of cells, and among the cationic barcodes that are contacted to a different subpopulation. In some embodiments, each of the two or more subpopulations of cells are contacted with one or more cationic barcodes before they are combined to form the population of cells. In some embodiments, contacting each of the two or more subpopulations of cells before they are combined to form the population of cells results in each subpopulation of cells having a different set of one or more cationic barcodes with unique sequences. In some embodiments, the two or more subpopulations of cells are combined in order to form the population of cells after the two or more subpopulations of cells have been contacted with one or more unique cationic barcodes. In some embodiments, the unique one or more cationic barcodes of each of the two or more subpopulations of cells of the population of cells are sequenced. In some embodiments, sequencing the unique one or more cationic barcodes of each of the two or more subpopulations of cells identifies individual cells as belonging to one subpopulation of cells among the two or more subpopulations of cells in the population of cells. In some embodiments, the individual cells are identified as belonging to one subpopulation of cells among the two or more subpopulations of cells by the sequences of the nucleic acid barcodes of the individual cells.

In some embodiments, the population of cells comprising two or more (e.g. at least 2, 3, 4, 5, 6, 7, 8, 9, 10 50, 100, 500, 1000) subpopulations of cells is an organoid. In some embodiments, the organoid is a liver organoid. In some embodiments, the population of cells comprising two or more (e.g. at least 2, 3, 4, 5, 6, 7, 8, 9, 10 50, 100, 500, 1000) subpopulations of cells is a liver organoid. In some embodiments, the organoid is formed from cells from a number of individuals that is, is about, is at least, is at least about, is not more than, or is not more than about, 2, 3, 4, 5, 6, 7, 8, 9, 10, 20, 30, 40, 50, 60, 70, 80, 90, 100, 200, 300, 400, 500, 600, 700, 800, 900, or 1000 individuals, or any number of individuals within a range defined by any two of the aforementioned numbers, for example 1 to 1000 individuals, 10 to 500 individuals, 50 to 100 individuals, 1 to 200 individuals, or 50 to 1000 individuals. In some embodiments, the organoid is formed from iPSCs, definitive endoderm, or foregut spheroids, or any combination thereof. In some embodiments, the organoid is formed from iPSCs, definitive endoderm, or foregut spheroids from cells from two or more individuals. In some embodiments, the organoid is formed from two or more subpopulations of cells, where the subpopulations of cells are iPSCs, definitive endoderm, or foregut spheroids. In some embodiments, the subpopulations of cells are synchronized. In some embodiments, each of the subpopulations of cells are contacted with one or more (e.g. at least 1, 2, 3, 4, 5) cationic barcodes before pooling and forming the organoid. In some embodiments, the organoid comprises two or more subpopulations comprising different cationic barcodes. In some embodiments, sequencing the cationic barcodes of the organoid identifies individual cells of the organoid as belonging to one of the two or more subpopulations of cells. In some embodiments, where the organoid is a liver organoid, the individual cells are further identified as hepatocytes, stellate cells, or biliary cells, or any combination thereof. In some embodiments, individual cells are identified based on expression of one or more (e.g. at least 1, 2, 3, 4, 5) of HNF4α, ASGR1, CEBPA, RBP4, COL1A2, SPARC, TAGLN, KRT7, TACSTD2, or SPPI, or any combination thereof.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden): ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, ESH1, 72.3, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

The liver is a vital organ that provides many essential metabolic functions for life such as the detoxification of exogenous compounds and coagulation as well as producing lipids, proteins, ammonium, and bile. Primary hepatocytes are a highly polarized metabolic cell type, and form a bile canaliculi structure with micro villi-lined channels, separating peripheral circulation from the bile acid secretion pathway. In vitro reconstitution of a patient's liver may provide applications including regenerative therapy, drug discovery and drug toxicity studies. Existing methodology using primary liver cells exhibit extremely poor functionality, largely due to a lack of essential anatomical structures, which limits their practical use for the pharmaceutical industry. The formation of liver organoids, which comprise a luminal structure with internalized microvilli and mesenchymal cells, as well as exhibit liver cell types such as hepatocytes, stellate cells, Kupffer cells, and liver endothelial cells, and methods of making and use thereof have previously been described in PCT Publications WO2018/085615, WO2018/085622, WO2018/085623, and WO2018/226267, each of which is hereby expressly incorporated by reference in its entirety.

In some embodiments, ESCs, germ cells, or iPSCs are cultured in growth media that supports the growth of stem cells. In some embodiments, the ESCs, germ cells, or iPSCs are cultured in stem cell growth media. In some embodiments, the stem cell growth media is RPMI 1640, DMEM, DMEM/F12, Advanced DMEM, hepatocyte culture medium (HCM), StemFit, mTeSR 1, or mTeSR Plus media. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors. In some embodiments, the stem cells are grown on a feeder cell substrate. In some embodiments, the stem cells are not grown on a feeder cell substrate. In some embodiments, the stem cells are grown on plates coated with laminin. In some embodiments, the stem cells are grown supplemented with FGF2 or a ROCK inhibitor (e.g. Y-27632), or both.

In some embodiments, the PSCs are cultured in feeder cell-free conditions. In some embodiments, the PSCs are cultured in mTeSR medium. In some embodiments, the PSCs are passaged upon reaching a confluency that is, is about, is at least, is at least about, is not more than, or is not more than about, 60%, 70%, 80%, 90%, or 100%. In some embodiments, the PSCs are cultured with a ROCK inhibitor and Laminin-511.

Any methods for producing definitive endoderm (DE) from pluripotent cells (e.g., iPSCs or ESCs) are applicable to the methods described herein. Exemplary methods are disclosed in, for example, U.S. Pat. No. 9,719,068. In some embodiments, iPSCs are used to produce definitive endoderm.

In some embodiments, one or more growth factors are used in the differentiation process from pluripotent stem cells to DE cells. In some embodiments, the one or more growth factors used in the differentiation process include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, or any combination thereof. In some embodiments, the PSCs are contacted with the one or more growth factors for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 60, 70, 80, 90, 100, 110, 120, 130, 140, 150, 160, 170, 180, 190, 200, 210, 220, 230, or 240 hours, or any number of hours within a range defined by any two of the aforementioned number of days, for example, 1 to 240 hours, 20 to 120 hours, 30 to 50 hours, 1 to 100 hours, or 50 to 240 hours. In some embodiments, the PSCs are contacted with the one or more growth factors at a concentration that is, is about, is at least, is at least about, is not more than, or is not more than about, 10, 20, 30, 40, 50, 60, 70, 80, 90, 100, 200, 300, 400, 500, 600, 700, 800, 900, or 1000 ng/mL, or any concentration within a range defined by any two of the aforementioned concentrations, for example, 10 to 1000 ng/mL, 50 to 800 ng/mL, 100 to 500 ng/mL, 10 to 200 ng/mL or 100 to 1000 ng/mL. In some embodiments, the concentration of the one or more growth factors is maintained at a constant level through the period of contacting. In some embodiments, the concentration of the one or more growth factors is varied during the period of contacting. In some embodiments, the one or more growth factors is dissolved into the growth media. In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

In some embodiments, the definitive endoderm cells are contacted with one or more modulators of a signaling pathway described herein. In some embodiments, the definitive endoderm cells are treated with the one or more modulators of a signaling pathway for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, hours, or 1, 2, 3, 4, 5, 6, 7, 8, 9, 10, 11, 12, 13, 14, 15, 16, 17. 18, 19, or 20 days, or any number of hours or days within a range defined by any two of the aforementioned number of days or hours, for example, 1 hour to 20 days, 20 hours to 10 days, 1 hour to 48 hours, 1 day to 20 days, 1 hour to 5 days, or 24 hours to 20 days. In some embodiments, the concentration of the one or more modulators of a signaling pathway is maintained at a constant level through the period of contacting. In some embodiments, the concentration of the one or more modulators of a signaling pathway is varied during the period of contacting.

In some embodiments, to differentiate the definitive endoderm into foregut spheroids, the definitive endoderm cells are contacted with one or more modulators of an FGF pathway and a Wnt pathway. In some embodiments, cellular constituents associated with the Wnt and/or FGF signaling pathways, for example, natural inhibitors, antagonists, activators, or agonists of the pathways can be used to result in inhibition or activation of the Wnt and/or FGF signaling pathways. In some embodiments, siRNA and/or shRNA targeting cellular constituents associated with the Wnt and/or FGF signaling pathways are used to inhibit or activate these pathways.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic, and FGF2 is also known as basic fibroblast growth factor (bFGF). Members FGF 11, FGF12, FGF13, and FGF14, also known as FGF homologous factors 1-4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects. In some embodiments, the FGF used is one or more (e.g. at least 1, 3, 5) of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, FGF23. In some embodiments, the FGF used is FGF4. In some embodiments, the definitive endoderm is contacted with an FGF at a concentration that is, is about, is at least, is at least about, is not more than, or is not more than about, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 ng/mL, or any concentration within a range defined by any two of the aforementioned concentrations, for example, 10 to 2000 ng/mL, 50 to 1500 ng/mL, 500 to 100 ng/mL, 10 to 1000 ng/mL or 500 to 2000 ng/mL.

In some embodiments, to differentiate the definitive endoderm into foregut spheroids, the definitive endoderm is contacted with a Wnt protein or activator. In some embodiments, the definitive endoderm is contacted with a glycogen synthase kinase 3 (GSK3) inhibitor. GSK3 inhibitor act to activate Wnt pathways. In some embodiments, the definitive endoderm is contacted with the GSK3 inhibitor Chiron (CHIR99021). In some embodiments, the definitive endoderm is contacted with CHIR99021 at a concentration that is, is about, is at least, is at least about, is not more than, or is not more than about, 0.1, 0.2, 0.3, 0.4, 0.5, 0.6, 0.7, 0.8, 0.9, 1, 2, 3, 4, 5, 6, 7, 8, 9, or 10 μM of CHIR99021 or any concentration within a range defined by any two of the aforementioned concentrations, for example, 0.1 to 10 M, 0.4 to 6 M, 1 to 5 μM, 0.1 to 1 μM, or 0.5 to 10 μM of CHIR99021.

In some embodiments, the foregut spheroids are differentiated into liver organoids. In some embodiments, the foregut spheroids are differentiated into liver organoids by contacting the foregut spheroids with retinoic acid (RA). In some embodiments, the foregut spheroids are contacted with RA at a concentration that is, is about, is at least, is at least about, is not more than, or is not more than about, 0.1, 0.2, 0.3, 0.4, 0.5, 0.6, 0.7, 0.8, 0.9, 1, 2, 3, 4, 5, 6, 7, 8, 9, or 10 μM of RA or any concentration within a range defined by any two of the aforementioned concentrations, for example, 0.1 to 10 μM, 0.4 to 6 M, 1 to 5 μM, 0.1 to 1 μM, or 0.5 to 10 μM of RA.

In some embodiments, one or more of the induced pluripotent stem cells, definitive endoderm, foregut spheroids, or liver organoid, or any combination thereof is prepared according to methods described in PCT Publications WO 2018/085615, WO 2018/191673, WO 2018/226267, WO 2019/126626, WO 2020/023245, WO 2020/056158, and WO 2020/069285, each of which is hereby expressly incorporated by reference in its entirety, and for the purposes of producing induced pluripotent stem cells, definitive endoderm, foregut spheroids, or liver organoids, or any combination thereof.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1. Synthesis and Characterization of POLY-Seq Polymers

A set of polymers was created using commercially available reagents to investigate the ability to tag cells with single-stranded DNA (ssDNA) barcodes in a ubiquitous manner to allow for rapid, cost-efficient multiplexing for single cell NGS techniques.

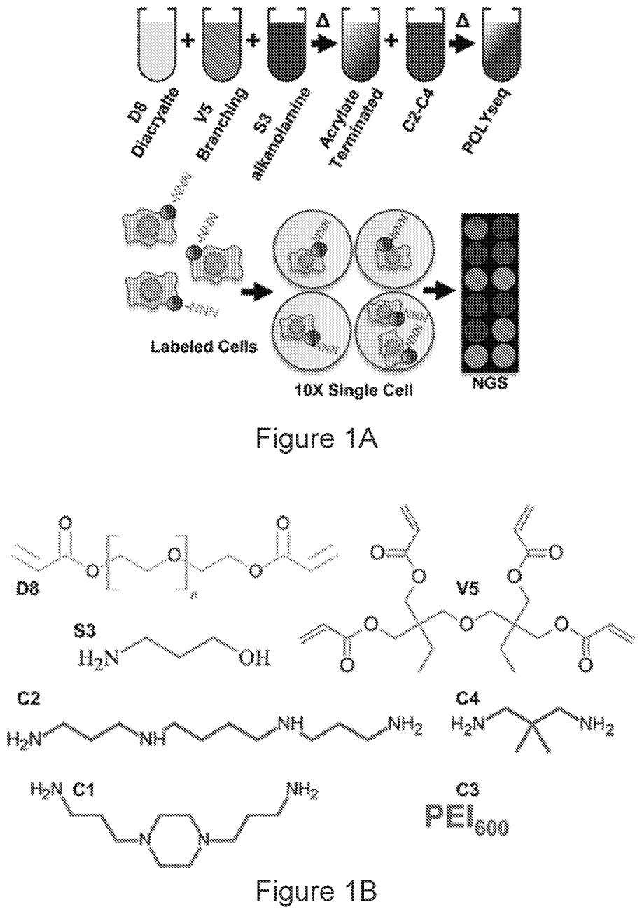

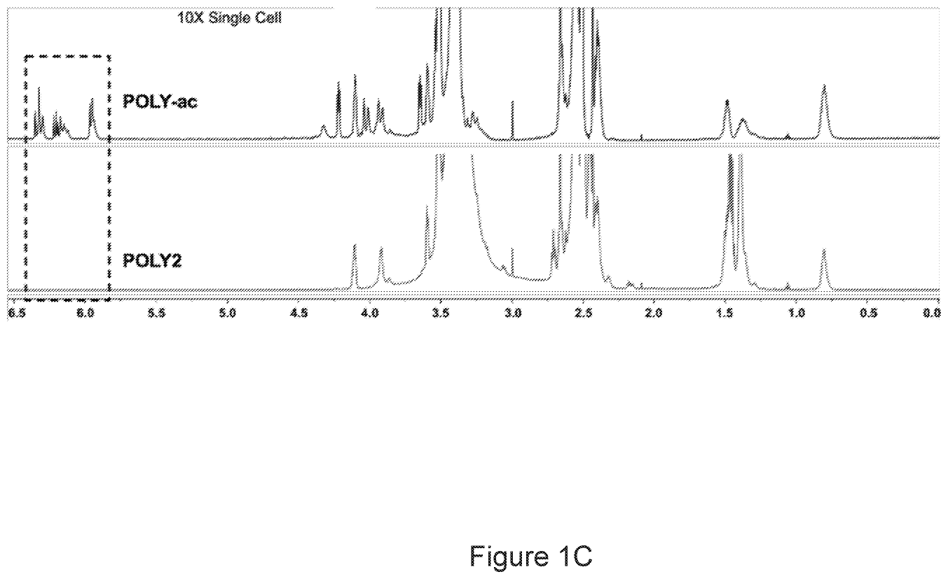

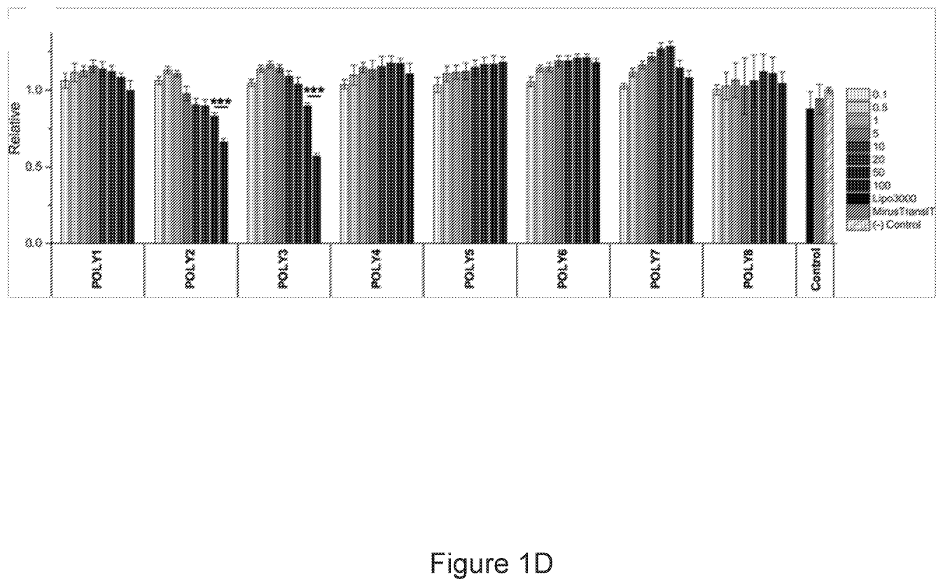

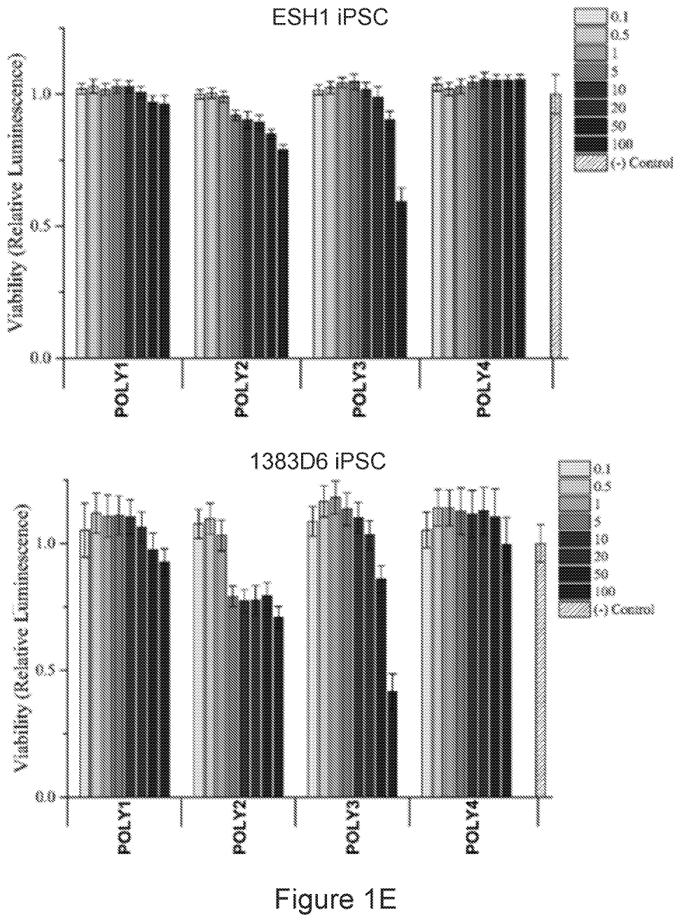

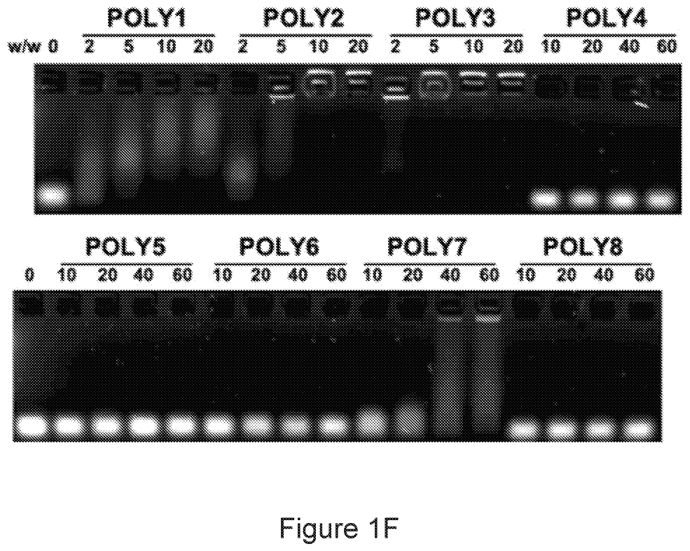

The synthesis and application scheme for POLY-seq vectors is detailed in FIG. 1A. Acrylate monomers mixed with an amino alcohol are heated to form the uncapped acrylate-terminated vector. Vectors are capped through the addition of a primary or secondary amine containing small molecule thereby imparting the ability for POLY-seq vectors to bind ssDNA barcodes and adhere to cells in a cell type independent manner (labeled cells). Labeled cells may then be processed using standard single cell techniques. All respective reagents are commercially available (FIG. 1B). ¹H NMR confirmed the presence of terminal acrylate groups following the production of the acrylate terminated products; resonant peaks for these groups were observed at S 6.2-5.6 and disappeared upon successful conjugation with capping reagents (FIG. 1C). Impact on cell viability was assessed using ESH1, 72.3 and 1383D6 iPSCs. An onset in the significant reduction of CTG luminescence beginning at 50 μg/mL, p<0.001, n=3, was found with polymers including branched V5 monomer with capping groups C2 and C3 (POLY2 and POLY3, respectively) (FIG. 1D). Results were recapitulated in ESH1 and 1383D6 iPSCs (FIG. 1E). To test the ability for capped vectors to bind and retain ssDNA barcodes, vectors and barcodes were initially mixed and allowed to bind in 25 mM HEPES pH 7.4 for 10 minutes. Following binding, vectors were loaded into a 2.5% agarose gel and run at 150 V. The ability to bind single-stranded DNA barcodes used in cell hashing experiments was found to be dependent upon capping reagent and backbone structure (FIG. 1F). Vectors capped with molecules C2 and C3 were found to be more readily retain ssDNA barcodes during gel electrophoresis than those capped with C1 or C4. Moreover, inclusion of branching acrylate V5 significantly reduced the mass ratio (w/w) at which complete barcode retention was observed (POLY2 vs POLY6, POLY3 vs POLY7).

### Example 2. POLY-Sea Vectors Target Cells Specifically

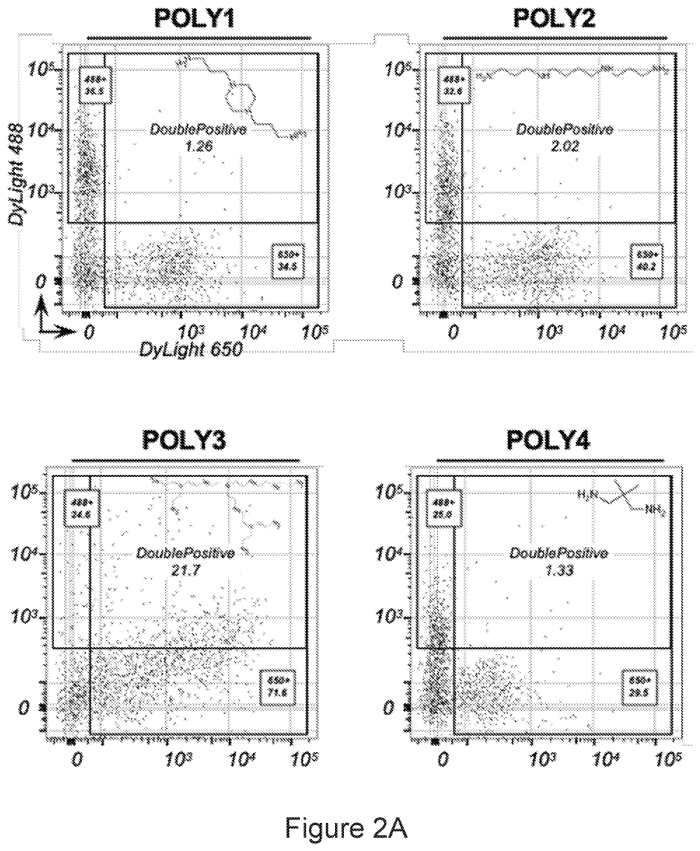

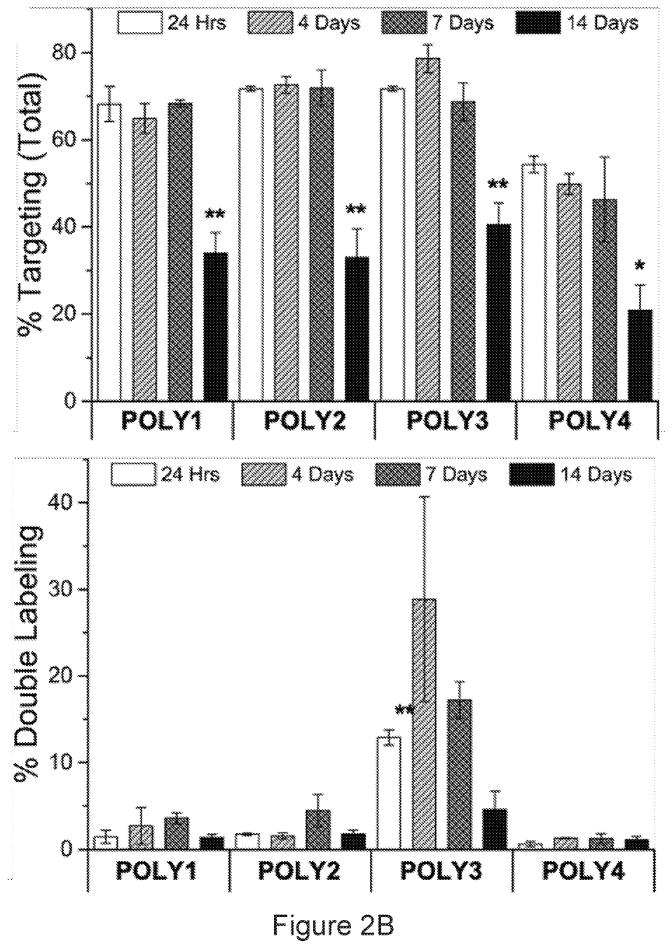

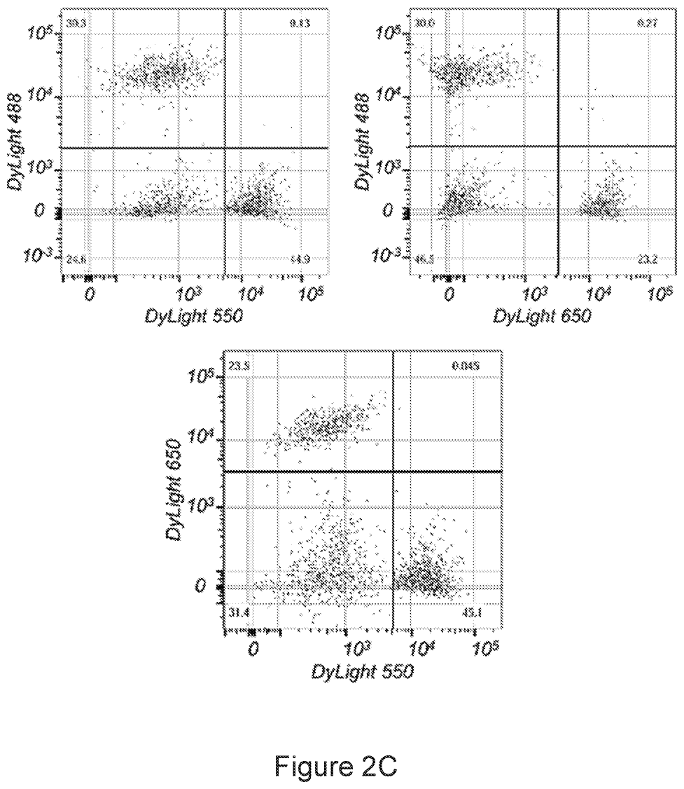

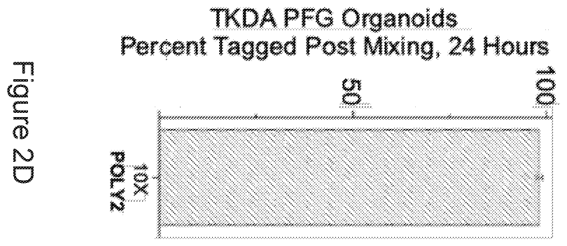

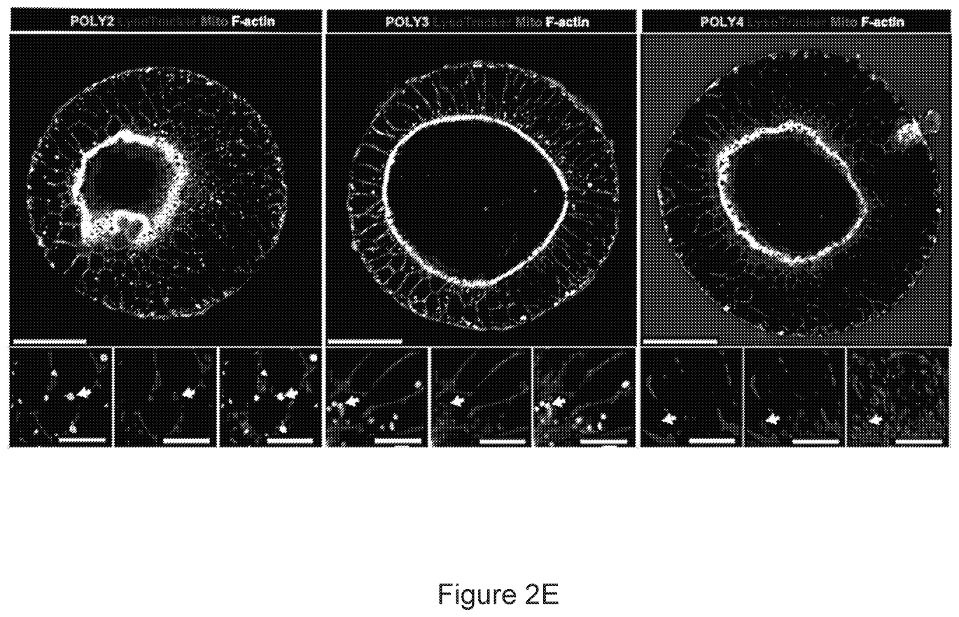

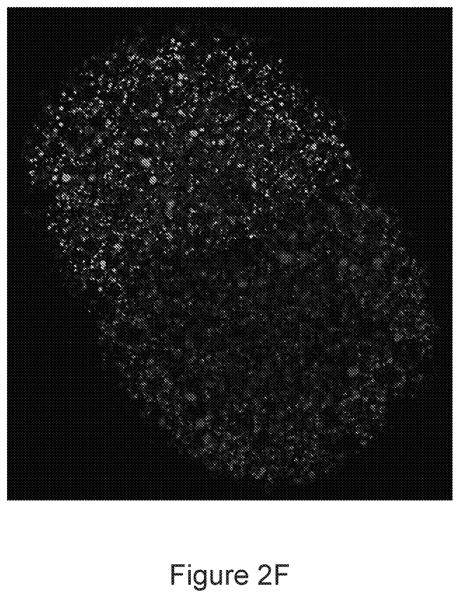

While an ability to rapidly bind and retain ssDNA barcodes is an important feature, vectors must also possess an ability to target cells. To this end, vectors POLY1-POLY4 were selected for quantification of cellular targeting. Targeting propensity of POLY-seq vectors was initially tested using FACS analysis of labeled anterior and posterior foregut spheroids. Gating analysis for day 4 isolated single cells is shown in FIG. 2A. Variance in extent of total labeling as well as double labeling was observed to be dependent on vector formulation (FIG. 2B). Significant reductions in total targeting percentage were observed at day 14 while no significant differences were found within the first 7 days of co-culture, indicating longevity of labeling fidelity. Vector POLY3 provided the greatest extent of double labeling and was significantly higher than POLY1, POLY2, and POLY4 beginning at the first time point (p<0.01, n=3) (FIG. 2B). Labeling fidelity is recapitulated by confocal imaging. Spheroids fused following labeling with POLY2 show distinct labeling with a visible boundary (FIG. 2F). Utility of vector POLY2 in binding human liver organoids was further examined using FACS analysis of isolated single cells from mixed cultures (FIG. 2C). Vector POLY2 was chosen based on performance in barcode binding and cellular targeting. Vector POLY2 had a total labeling percentage of 98.2±0.8% of cells isolated from HLO cultures (FIG. 2D). Double labeled cells within this mixed culture by FACS analysis was negligible. For investigation into the spatial distribution of cell-bound POLY-seq vectors, DyLight 488 conjugated vectors were incubated with HLO cultures. Confocal analysis revealed strong colocalization with lysosomes for POLY2 and POLY3 while POLY4 had comparatively lower internalization at three houses, mirroring weaker labeling found by flow cytometry (FIG. 2E). These results suggest a correlation between each vector's ability to bind barcodes and interact with cells.

### Example 3. POLY-Seq Vectors Deliver Amplifiable Barcodes

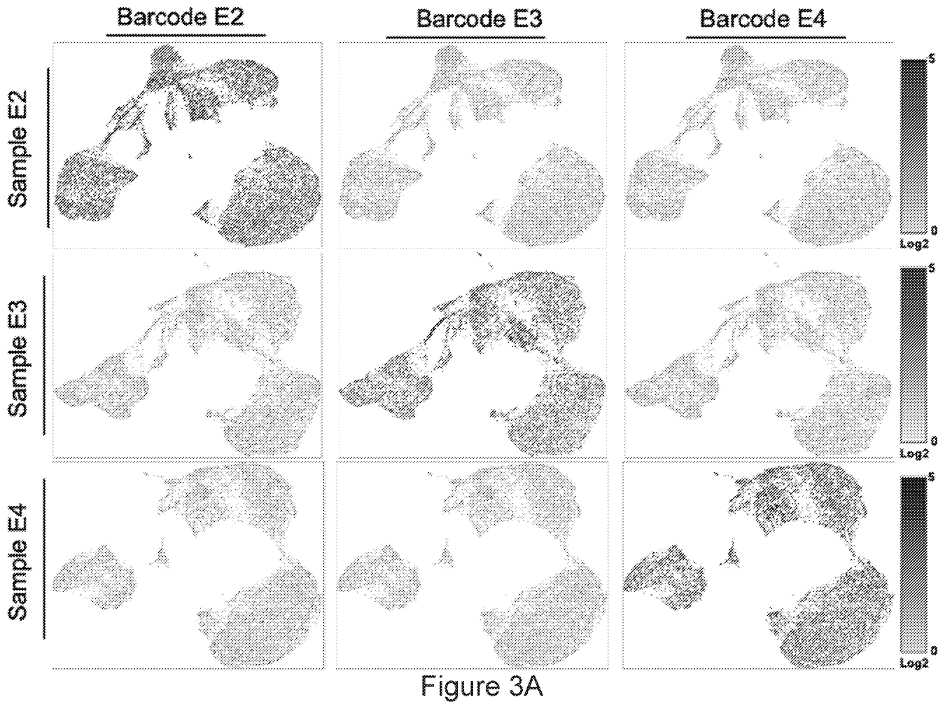

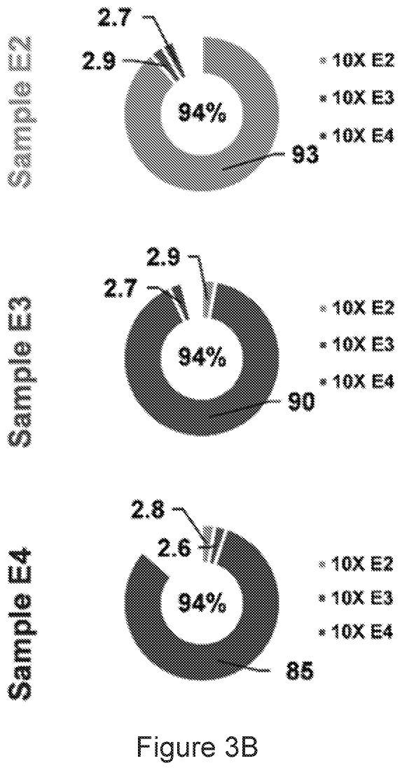

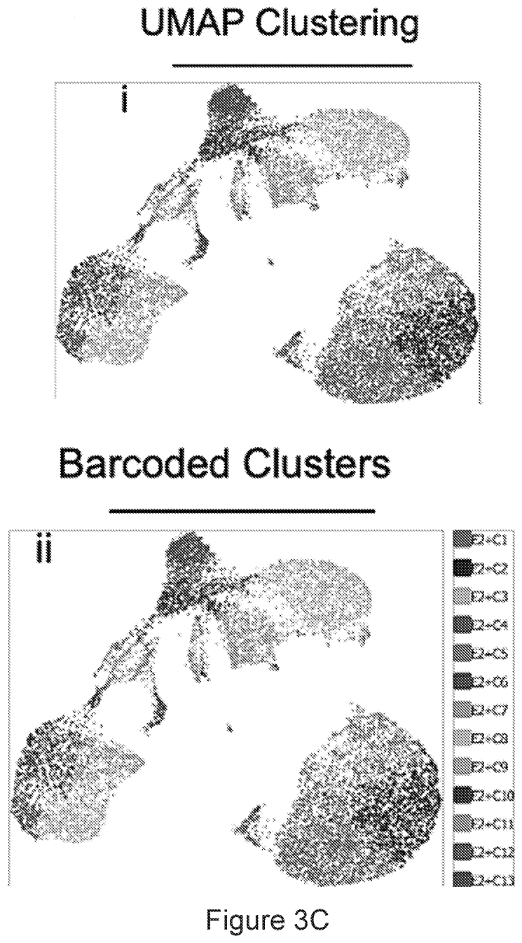

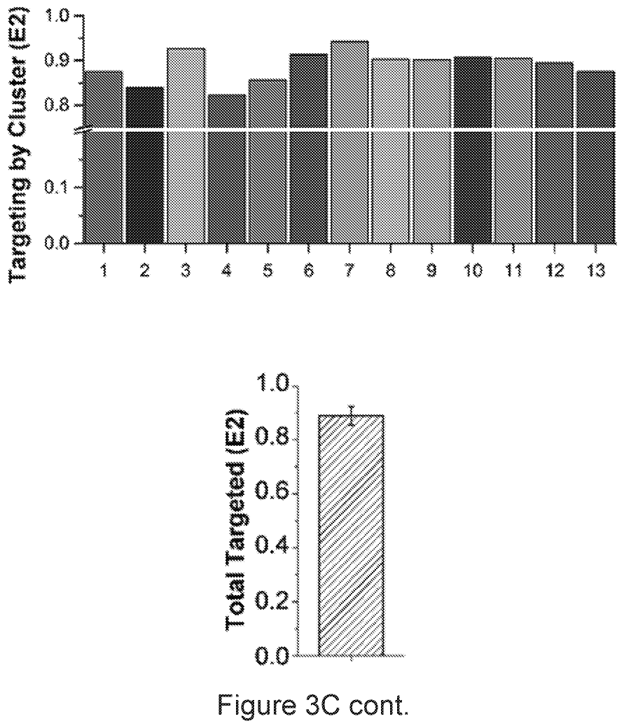

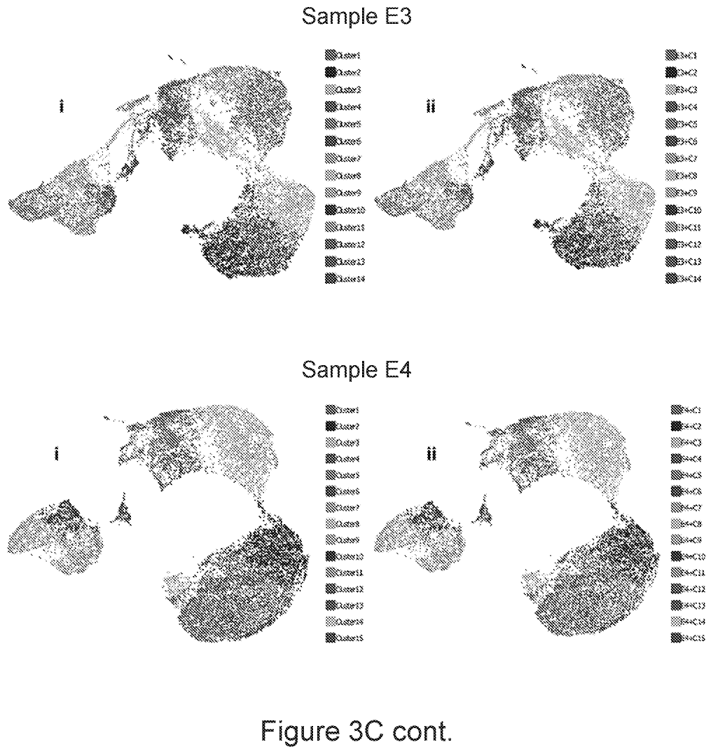

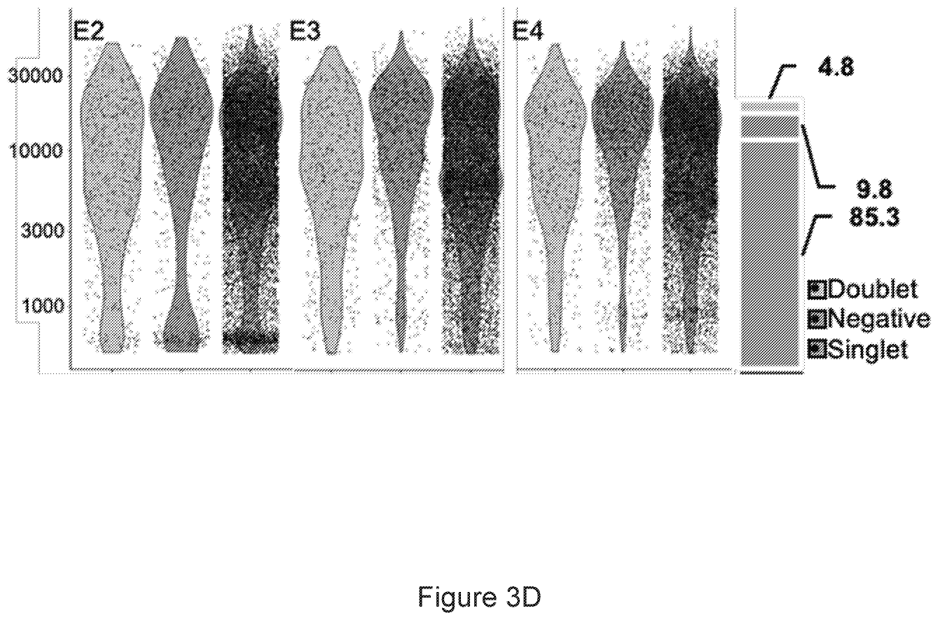

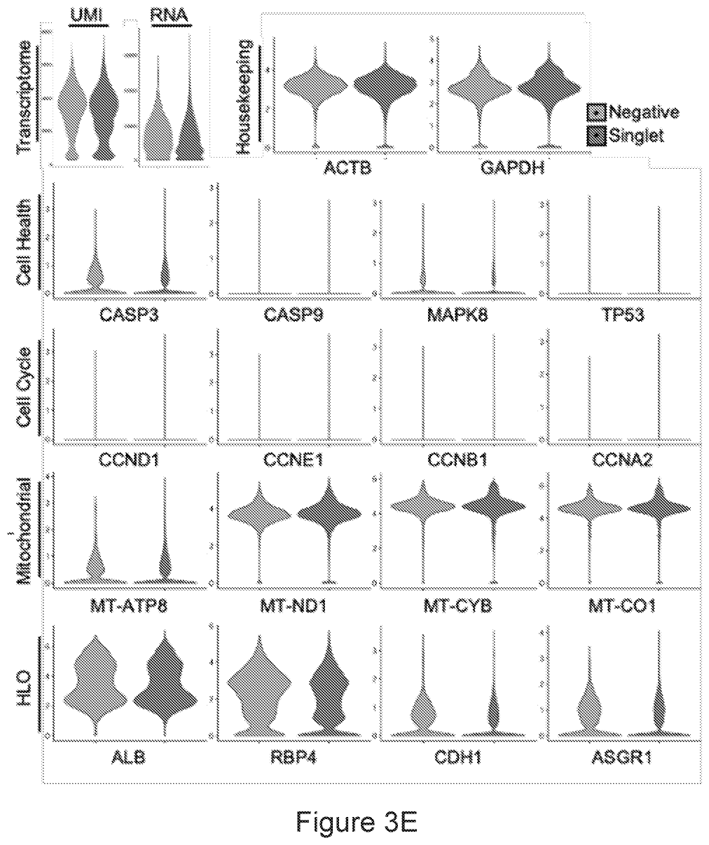

To test the ability for POLY-seq vectors to deliver barcodes which may be amplified by the standard 10× Chromium workflow and read by common next-generation sequencers, three HLO samples were individually tagged with three distinct barcodes using vector POLY2 for one hour prior to being run on the 10× Chromium platform. Single-cell analysis of barcoded HLOs containing all sequenced barcodes revealed a high extent of labeling across the three populations with a total extent of labeling near 90%, reflecting targeting percentages observed during initial FACS analysis (FIGS. 3A-B). Sequencing accuracy for all three barcodes was 94%. Importantly, uniformity of labeling across multiple clusters was verified by UMAP analysis using a high clustering sensitivity, indicating unbiased labeling. All cells for sample E2 were grouped into 13 clusters and juxtaposed with cells only containing the correct barcode read (FIG. 3C). Analysis for samples E3 and E4 were similarly performed (FIG. 3C). Barcoding uniformity across clusters was confirmed for all three samples with average labeling per cluster for samples E2, E3, and E4 found to be 89±3.4%, 86±4.8%, and 81±5.9%, respectively (FIG. 3D). This reduction in labeling percentage by single-cell sequencing compared with flow analysis is attributed to the reduced labeling time used during single-cell preparation (1 vs 24 hours) and provides the opportunity to directly assess potential impacts of POLY2 labeling on measured gene expression by DESeq2. Perturbation to measured transcription by labeling was examined using singlet and negative-labeled cells; both populations were compared using an array of genes: housekeeping (ACTB, GAPDH, PGK1), cell health, associated with autophagy and apoptosis (CASP3, CASP9, MAPK8, TP53), cell cycle cyclins (CCND1, CCNE1, CCNB1, CCNA2), mitochondrial (MT-ATP8, MT-ND1, MT-CYB, MT-CO1), and human liver organoid (ALB, RBP4, CDH1, ASGR1). Labeling was found not to alter transcriptome expression amongst these populations (FIG. 3E, Table 1).

TABLE 1
Adjusted p-values for listed genes comparing barcoded
Singlet vs Negative samples by DESeq2. CCNA2 expression
was not detected by DE processing (n.d.)

| Category | Gene | Adjusted p-value |
|---|---|---|
| Housekeeping | ACTB | 1 |
| | GAPDH | 0.37 |
| Cell Health | CASP3 | 1 |
| | CASP9 | 1 |
| | MAPK8 | 1 |
| | TP53 | 1 |
| Cell Cycle | CCND1 | 1 |
| | CCNE1 | 1 |
| | CCNB1 | 1 |
| | CCNA2 | n.d. |
| Mitochondrial | MT-ATP8 | 1 |
| | MT-ND1 | 1 |
| | MT-CYB | 1 |
| | MT-CO1 | 1 |
| HLO | ALB | 1 |
| | RBP4 | 1 |
| | CDH1 | 1 |
| | ASGR1 | 1 |

### Example 4. POLY-Seq Barcoding Identifies Multiple Population Lineages in HLOs

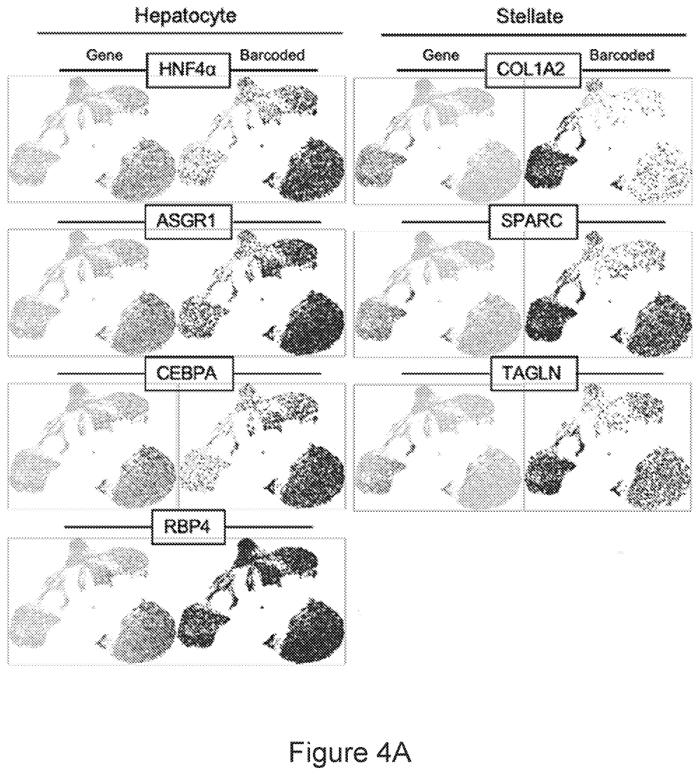

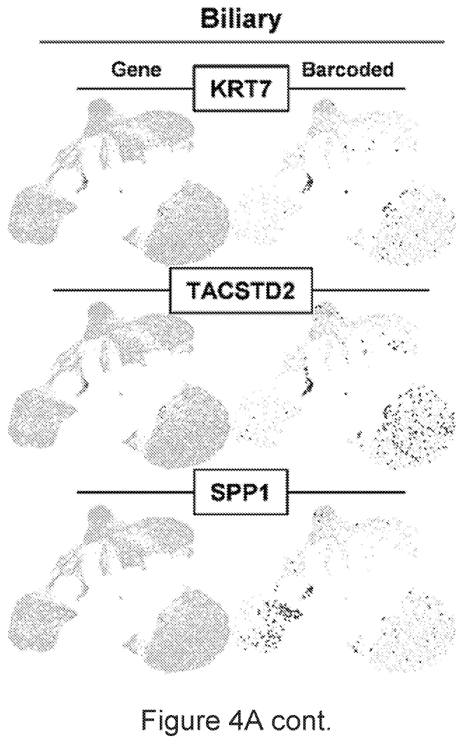

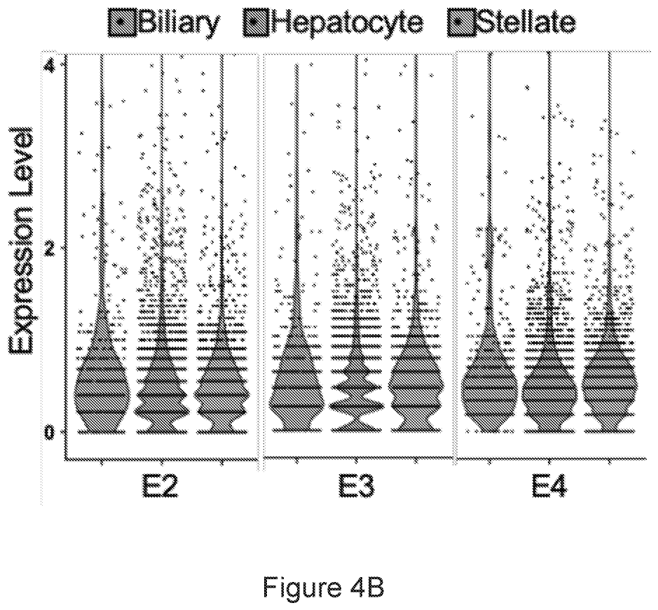

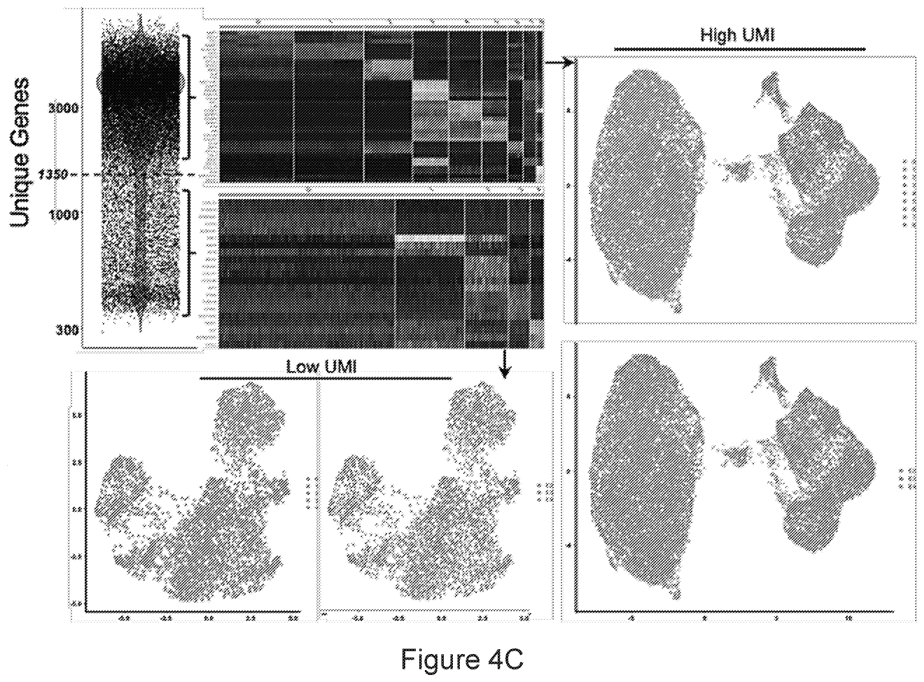

As multicellularity has been demonstrated in the HLO culture system, heterogenous barcoding potential was further demonstrated through HLO lineage identification. Hepatocytes, identified by hepatocyte nuclear factor 4 alpha (HNF4α), asialoglycoprotein receptor 1 (ASGR1), CCAAT enhancer binding protein alpha (CEBPA), and retinol binding protein 4 (RBP4); stellate cells, identified by collagen type 1, alpha 2 (COL1A2), secreted protein acidic and cysteine rich (SPARC), and transgelin (TAGLN); and biliary cells identified by keratin 7 (KRT7), epithelial glycoprotein-1 (TACSTD2), and secreted phosphoprotein 1 (SPP1), possessed a significant degree of representation amongst the barcoded population (FIG. 4A). Barcode representation was examined and found to be uniformly expressed within these populations (FIG. 4B). Finally, the ability for POLY-seq to successfully barcode cells through a wide range of expressed unique genes, single-labeled cells were split into high and low UMI fractions with a cut-off of 1350 similar to previous analyses (FIG. 4C). Seurat clustering distinctly identified populations amongst both fractions. High and low UMI fractions were highly represented by POLY-seq barcodes with an average of 83±4.7% and 88±4.6% of the populations identified as single-labeled cells, respectively, mirroring previous barcoding performance using lipid-based methods.

### Example 5. Observations of the POLY-Seq Technique

As disclosed herein, cationic polymers were prepared as vectors capable of binding nucleic acids for delivery. Polymers were synthesized through Michael Addition using commercially available acrylate terminated monomers and alkanolamines. Vectors POLY2 and POLY3 showed a significant reduction in CTG luminescence beginning at concentrations of 50 μg/mL over a time period of 24 hours (p<0.001) while neither POLY1 nor POLY4 showed any appreciable perturbation to viability over the concentrations tested (FIG. 1D, E), serving as a reference point to understand potential toxicity from long-term labeling. To successfully deliver nucleic acids into cells, a vector must possess at least two properties: the ability to retain bound DNA/RNA and the ability to bind, and remain bound to cells for some appreciable amount of time. The ability for POLY-seq vectors to rapidly bind and retain nucleic acids such as CITE-seq hashing ssDNA barcodes, for single cell applications was examined using gel electrophoresis. Those vectors with branching acrylate monomers (V5) and capped with monomers containing a high density of primary and secondary amines (C2, C3) most readily bound and retained ssDNA barcodes under physiological pH. Onset of complete binding for vectors POLY2 and POLY3 as indicated by the reversal of DNA migration was observed at w/w=10 and 5, respectively. Conversely, vectors created exclusively with diacrylate monomer D8 and alkanolamine S3 (POLY5-POLY8) showed a drastic reduction in binding activity (FIG. 1F). Success of ssDNA binding is therefore a combination of branching architecture and cap type. As vectors created with branching acrylates (POLY1-POLY4) showed a greater propensity for binding ssDNA, these variants were chosen for further investigation into cell targeting.

Quantification of cell targeting was achieved using flow cytometry to track fluorescently labeled vectors in a model anterior/posterior gut boundary fusion system. Percent cellular labeling between vectors POLY1-POLY3 were not significantly different within the first seven days, suggesting binding fidelity. While vector POLY3 provided the highest extent of total labeling, it showed a significant degree of double labeling juxtaposed with the other three vectors at all time points. Interestingly, while vector POLY4 was unable to retain ssDNA barcodes when subject to electrophoresis, it showed an ability to associate with cells. Based on ssDNA binding efficiency and cell targeting performance, POLY2 was considered the main candidate for single-cell barcoding applications of human liver organoid (HLO) cultures. FACS analysis revealed that nearly all cells from HLO samples were tagged with POLY2 with no appreciable double labeling 24 hours after mixing of individually tagged cultures. Confocal analysis of fluorescent conjugated POLY-seq revealed formulation dependent colocalization within lysosomes three hours after incubation with the culture system. As lysosomal sequestration is generally associated with maturation or fusion of late endosomes from early endosomes trafficked from clathrin-dependent, dynamin-dependent endocytosis or micropinocytosis, it suggests that cellular association of vector POLY2 and POLY3 readily occurs prior to this time point. Although the internalization mechanism is molecularly unknown, this selective association provides investigative opportunities into time-dependent endosomal/lysosomal organelle trafficking.

Apart from possessing an ability to bind barcodes and tagging cells, functional delivery of ssDNA barcodes by some system ultimately relies upon readable, unique sequences correctly captured and amplified by single cell preparation techniques for the system to even be considered useful. The polymer vectors described herein had efficient qualities for barcode binding, cellular labeling and retention, and delivered readable barcodes which can be identified during scRNA-seq after one hour of labeling in-situ in a highly uniform manner. Juxtaposing cells without barcodes (Negative) and single-labeled cells (Singlet), no difference was found in the distribution of the number of unique genes (UMI) or total RNA per cell as well as general transcriptome expression. This suggests that POLY-seq barcoding does not interfere with single-cell library preparation and analysis nor perturbs cellular physiology at the transcript level. Moreover, POLY-seq uniformly labeled heterogeneous populations, quantified as both labeling percentage and barcode expression. A cost estimate for synthesizing vector POLY2 is 3 cents/mg. 10 μg were used per HLO sample. With specific intracellular vesicle sequestration, the ability to fluorescently label, and to rapidly bind and deliver ssDNA barcodes into cells without the need for covalent conjugation, the POLY-seq system provides the opportunity to inexpensively generate custom barcoded pools for multiplex applications, saving considerable time and sequencing costs.

### Example 6. Materials and Methods

Synthetic Materials:

The following materials were purchased from Sigma-Aldrich and used without further purification: Poly(ethylene glycol) diacrylate, Mₙ=250 ≥92%; Di(trimethylolpropane) tetraacrylate; 3-amino-1-propanol ≥99%; 1,4-Bis(3-aminopropyl)piperazine ≥99%; spermine ≥99%, polyethylenimine, Mₙ=600; 2,2-dimethyl-1,3-propanediamine ≥99%; DMSO ≥99%; DMSO-d₆ 99.9% atom % D, containing 0.03% (v/v) TMS.

Polymer Synthesis:

POLY-seq vectors were synthesized through Michael Addition in a two-step process with reagents tabulated herein. Acrylate terminated monomers, alkanolamine monomers, and capping agents were initially dissolved in anhydrous DMSO at 200 mg/mL. Reagents were homogeneously mixed in glass 12×75 mm culture tubes at defined ratios and allowed to react at 90° C. for 20 hours to form the acrylate terminated product (POLY-ac). Temperature was held constant using a silicone oil bath. Amine conjugation of terminal acrylate groups was achieved in the second step through the addition of capping agents. Terminal acrylate conjugation was allowed to continue at 50° C. for 24 hours to generate the final POLY-seq polymer vectors (Table 2). Aliquots of the final products were maintained at −20° C. for long term storage. Dissolution of the polymers for application testing was achieved by direct dilution of the concentrated DMSO stock into 25 mM HEPES buffer, pH 7.4, at a final concentration of 1 and 10 mg/mL. All DyLight reagents were dissolved in DMSO to a final concentration of 10 mg/mL. DyLight conjugation was achieved through mixing NHS-activated DyLight fluorescent molecules with 10 mg/mL POLY-seq vectors under vortex to a final concentration of 40 μg DyLight per 1 mg polymer.

List of acrylate, amine monomers, and capping molecules:

Acrylate Monomers: Poly(ethylene glycol) diacrylate, Mₙ=250 (“D8”); Di(trimethylolpropane) tetraacrylate (“V5”).

Alkanolamine: 3-amino-1-propanol (“S3”)

Capping Molecules: 1,4-Bis(3-aminopropyl)piperazine (“C1”), spermine (“C2”), polyethylenimine, Mₙ=600 (“C3”), 2,2-dimethyl-1,3-propanediamine (“C4”).

TABLE 2
POLY-seq polymer (vector) formulations

| POLY-seq | | | Acrylate |
|---|---|---|---|
| nomenclature | | D8:V5:S3 | Polymer:Capping |
| (Synthesis | | or D8:S3 | Molecule |
| number) | Formulation | (Molar ratio) | (Mass Ratio) |
| POLY1 (207) | D8 V5 S3 C1 | 1.05:0.7:1 | 100:75 |
| POLY2 (208) | D8 V5 S3 C2 | 1.05:0.7:1 | 100:75 |
| POLY3 (209) | D8 V5 S3 C3 | 1.05:0.7:1 | 150:250 |
| POLY4 (210) | D8 V5 S3 C4 | 1.05:0.7:1 | 100:50 |
| POLY5 (215) | D8 S3 C1 | 1.1:1 | 100:10 |
| POLY6 (216) | D8 S3 C2 | 1.1:1 | 100:10 |
| POLY7 (217) | D8 S3 C3 | 1.1:1 | 100:20 |
| POLY8 (218) | D8 S3 C4 | 1.1:1 | 100:10 |

NMR:

NMR was performed on a Bruker Ascend 600 MHz spectrometer. An aliquot of 5 mg of either acrylate terminated or capped vectors were directly dissolved in deuterated DMSO-d6 for sample acquisition. Free induction decay files were processed in Mnova.

Cell Culture/Toxicity:

Human embryonic stem cell clone H1 was provided by the WiCell Institute. iPSC clone 1383D6 was kindly gifted by Kyoto University. iPSC clone 72.3 was provided by the CCHMC Pluripotent Stem Cell Facility. Stem cells were maintained according to protocols known in the art with slight modifications, or as described herein. All stem cells were maintained in feeder cell-free conditions using mTeSR (Stem Cell Technologies) at 37° C. in 5% CO₂. Cells were passaged upon reaching 70% confluency by Accutase (Thermo Fisher) isolation and plated overnight in 6-well Falcon (Corning) plates with a supplement of 10 μg/mL Y-27632 (ROCK inhibitor) and 5 μg/mL Laminin-511. Y-27632/Laminin-511-supplemented mTeSR medium was changed to mTeSR along following overnight attachment and was changed with fresh mTeSR medium daily.

Toxicity screening was performed in white 96-well plates (Corning). A single cell suspension from passage plates was isolated using Accutase. Cells were plated into individual wells in mTeSR supplemented with Y-27632 and Laminin-511 as per maintenance at an initial concentration of 20,000 cells/well and maintained in mTeSR until reaching 80-90% confluency. POLY-seq polymers were diluted in mTeSR and applied to the cells for 24 hours. Viability was determined by the ATP-based CellTiter-Glo (CTG) 3D viability assay (Promega).

Flow Cytometry:

Anterior and posterior gut cultures were grown according to methods known in the art or as described herein. Following lineage establishment, cultures were then tagged by DyLight-conjugated POLY-seq vectors overnight at a concentration of 20 μg/mL with anterior and posterior gut cultures each receiving a distinct DyLight color (488 nm for anterior and 650 nm for posterior). Following tagging, cells were washed twice in DMEM/F-12 (Thermo Fisher) to remove unbound POLY-seq vector. Single cell suspensions were isolated and plated into ultra-low attachment U-bottom 96-well plates at an amount of 20,000 cells per well in mTeSR supplemented with Y-27632 and Laminin-511. Plates were briefly centrifuged at 160×g for 2 minutes to pellet cells. Spheroids were allowed to form overnight. Following formation, single spheroids tagged with POLY-seq-DyLight 488 were plated with single spheroids tagged with POLY-seq-DyLight 650 and allowed to fuse overnight. Fused spheroids were maintained as previously described. At 1, 4, 7, and 14 days post fusion, spheroids were digested using a mixture of 0.9× Accutase+1.0× TrypLE Express at 37° C. with gentle pipetting. Extent of total and double labeling were quantified using flow cytometry.

HLO Culture

Human hepatic liver organoids (HLOs) were generated according to methods known in the art with slight modification, or as described herein. For endoderm establishment, iPSCs were seeded into 6-well plates (Corning) in mTeSR supplemented with Y-27632 and Laminin-511. Medium was changed to mTeSR alone the following day. Medium was switched to RPMI-1640 (Life Technologies) containing 100 ng/mL Activin A (R&D Systems) and 50 ng/mL bone morphogenetic protein 4 (BMP4; R&D Systems) on the second day. This constitutes day 1 (D1) of differentiation. Medium was switched to RPMI-1640+100 ng/mL Activin A+0.2% KnockOut Serum Replacement (KOSR; Thermo Fisher) on day 2 (D2). Medium was switched to RPMI-1640+100 ng/mL Activin A+2.0% KOSR on day 3 (D3). Medium was switched to Advanced DMEM/F12+B27 (Life Technologies)+N2 (Gibco)+500 ng/mL fibroblast growth factor 4 (FGF-4; R&D Systems) and 3 μM CHIR99021 (R&D Systems) for days 4-6 (D4-6), changed daily. A single cell suspension was isolated on D7 using Accutase. Cells were washed and resuspended in growth factor Matrigel at 50,000 cells/50 μL of Matrigel. Into 6-well plates (VWR) were plated 50 μL drops. Medium was switched to Enrichment Medium (EP): Advanced DMEM/F12 (Gibco)+2% B-27 (Gibco)+1% N2 (Gibco)+1% HEPES (1M, Gibco)+1% Pen/Strep (Thermo Fisher)+1% L-glutamine (Thermo Fisher)+3 μM CHIR99021 (R&D Systems)+5 ng/mL FGF2 (R&D Systems)+10 ng/mL VEGF (Life Technologies)+20 ng/mL EGF (R&D Systems)+0.5 μM A83-01 (Tocris)+50 μg/mL ascorbic acid (Sigma) for D7-10, changed on D7 and D9. Medium was switched to Advanced DMEM/F12+2% B-27+1% N2+1% HEPES (IM)+1% Pen/Strep+1% L-glutamine+2 μM retinoic acid (Sigma) for D11-14, changed on D11 and D13. Medium was switched to hepatocyte culture medium (HCM; Lonza)+10 ng/mL hepatocyte growth factor (HGF; Peprotech)+Oncostatin M and changed every other day. HLOs were used between D21-D24. HLOs were individually tagged with POLY-seq vectors conjugated with either DyLight 488, 550, or 650 overnight in HCM, washed twice, and mixed for 24 hours prior to flow analysis. Mixed cultures were digested using a mixture of 0.9× Accutase+1.0× TrypLE Express at 37° C. with gentle pipetting. Extent of total and double labeling were quantified using flow cytometry.

Immunofluorescence:

HLOs were incubated with DyLight conjugated POLY-seq vectors diluted in HCM for 1-24 hours prior to live imaging. F-actin staining was achieved using SiR-Actin (Cytoskeleton, Inc.) at a concentration of 250 nM for three hours or 500 nM for one hour. Mitochondria were stained using Tetramethylrhodamine, methyl ester (TMRM; Thermo Fisher) at a concentration of 1 μM for a minimum of one hour. Lysosomes were stained with LysoTracker Blue DND-22 (Thermo Fisher) at a concentration of 1 μM for a minimum of one hour.

Cell Tagging for 10× Genomics Sequencing:

POLY2 was mixed with 10× compatible DNA barcoding oligomers based off of the CITE-seq cell hashing oligomer structure (Table 3), synthesized by Integrated DNA Technologies, at a mass ratio of 10 μg vector/1 μg oligo. 10 μg of POLY2 was first diluted in 50 μL of HCM with 1 μg of barcoding oligo diluted in a separate 50 μL aliquot. Barcoding oligo was quickly mixed by pipetting into POLY2 directly after dilution and allowed to stand undisturbed for 10 minutes to form the ready-to-use POLY-seq vector; the vector was then diluted into HLO aliquots to a final concentration of 10 μg vector/500 μL HCM. HLOs were tagged at 37° C. for one hour. HLOs were washed twice to remove barcoding vector from the supernatant and passaged into single cells by a mixture of Accutase/TrypLE Express (Gibco). Single cell suspensions were cleared of debris through a 40 μM filter and adjusted to a final concentration of 1000 cells/μL in HCM prior to loading into the Chromium chip and processed according to the Chromium Single Cell 3′ Reagent Kits v3 by 10× Genomics. Barcodes were amplified using a 3′ phosphorothioate stabilized additive primer with sequence: 5′-GTGACTGGAGTTCAGACGTGTGC*T*C-3′ (SEQ ID NO: 1). Following cDNA amplification, barcode sequences were separated from full-length mRNA-derived cDNA per the CITE-seq protocol and PCR amplified using standard P5/P7 adaptors containing an i7 index. Prepared scRNA-seq libraries were run on the NovaSeq 6000 system. Isolated barcode libraries were run separately on the NextSeq 550 system. Cellranger was used to align scRNA-seq reads to hg19 human genome and integrate barcode reads. Uniform manifold approximation and projection (UMAP) creation, cluster, and barcode expression were performed in Loupe offered by 10× Genomics. Identification of singlets/doublets was done using Seurat v3.1 pre-filtering cells to exclude those with transcriptomes composed of >25% mitochondrial counts and include cells with a number of uniquely identified genes between 100-10,000. Transcriptome differential expression was calculated in Seurat using DESeq2 (Bioconductor v3.11) using a log₂(1.1) fold-change pre-filter and 1000 cells per subsample.

TABLE 3
Single-stranded DNA oligonucleotide
barcoding sequences

| | | Sequence |
|---|---|---|
| Barcode | Sequence | number |
| E2 | 5′-GTGACTGGAGTTCAGACGTGTGCTCTTC- | SEQ ID |
| | CGATCTCATCTTGTGATCB(A)₃₀-3′ | NO: 2 |
| E3 | 5′-GTGACTGGAGTTCAGACGTGTGCTCTTC- | SEQ ID |
| | CGATCTAGAAGGACGAGTB(A)₃₀-3′ | NO: 3 |
| E4 | 5′-GTGACTGGAGTTVAGACGTGTGCTCTTC- | SEQ ID |
| | CGATCTCACCATGTACCAB(A)₃₀-3′ | NO: 4 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Shapiro, E., Biezuner, T. & Linnarsson, S. 2013, “Single-cell sequencing-based technologies will revolutionize whole-organism science”, Nature Reviews Genetics, vol. 14, no. 9, pp. 618-630.
Picelli, S., Björklund, Å K., Faridani, O. R., Sagasser, S., Winberg, G. & Sandberg, R. 2013, “Smart-seq2 for sensitive full-length transcriptome profiling in single cells”, Nature methods, vol. 10, no. 11, pp. 1096-1098.
Navin, N., Kendall, J., Troge, J., Andrews, P., Rodgers, L., McIndoo, J., Cook, K., Stepansky, A., Levy, D. & Esposito, D. 2011, “Tumour evolution inferred by single-cell sequencing”, Nature, vol. 472, no. 7341, pp. 90.
Buettner, F., Natarajan, K. N., Casale, F. P., Proserpio, V., Scialdone, A., Theis, F. J., Teichmann, S. A., Marioni, J. C. & Stegle, O. 2015, “Computational analysis of cell-to-cell heterogeneity in single-cell RNA-sequencing data reveals hidden subpopulations of cells”, Nature biotechnology, vol. 33, no. 2, pp. 155.
Zheng, G. X., Terry, J. M., Belgrader, P., Ryvkin, P., Bent, Z. W., Wilson, R, Ziraldo, S. B., Wheeler, T. D., McDermott, G. P. & Zhu, J. 2017, “Massively parallel digital transcriptional profiling of single cells”, Nature communications, vol. 8, no. 1, pp. 1-12.
Stoeckius, M., Hafemeister, C., Stephenson, W., Houck-Loomis, B., Chattopadhyay, P. K., Swerdlow, H., Satija, R & Smibert, P. 2017, “Simultaneous epitope and transcriptome measurement in single cells”, Nature methods, vol. 14, no. 9, pp. 865.
Stoeckius, M., Zheng, S., Houck-Loomis, B., Hao, S., Yeung, B. Z., Mauck, W. M., Smibert, P. & Satija, R. 2018, “Cell Hashing with barcoded antibodies enables multiplexing and doublet detection for single cell genomics”, Genome biology, vol. 19, no. 1, pp. 1-12.
Weber, R. J., Liang, S. I., Selden, N. S., Desai, T. A. & Gartner, Z. J. 2014, “Efficient targeting of fatty-acid modified oligonucleotides to live cell membranes through stepwise assembly”, Biomacromolecules, vol. 15, no. 12, pp. 4621-4626.
McGinnis, C. S., Patterson, D. M., Winkler, J., Conrad, D. N., Hein, M. Y., Srivastava, V., Hu, J. L., Murrow, L. M., Weissman, J. S. & Werb, Z. 2019, “MULTI-seq: sample multiplexing for single-cell RNA sequencing using lipid-tagged indices”, Nature methods, vol. 16, no. 7, pp. 619.
Kang, H. M., Subramaniam, M., Targ, S., Nguyen, M., Maliskova, L., McCarthy, E., Wan, E., Wong, S., Byrnes, L. & Lanata, C. M. 2018, “Multiplexed droplet single-cell RNA-sequencing using natural genetic variation”, Nature biotechnology, vol. 36, no. 1, pp. 89.
Guo, C., Kong, W., Kamimoto, K., Rivera-Gonzalez, G. C., Yang, X., Kirita, Y. & Morris, S. A. 2019, “CellTag Indexing: genetic barcode-based sample multiplexing for single-cell genomics”, Genome biology, vol. 20, no. 1, pp. 90.
Kebschull, J. M., da Silva, P. G., Reid, A. P., Peikon, I. D., Albeanu, D. F. & Zador, A. M. 2016, “High-throughput mapping of single-neuron projections by sequencing of barcoded RNA”, Neuron, vol. 91, no. 5, pp. 975-987.
Kebschull, J. M. & Zador, A. M. 2018, “Cellular barcoding: lineage tracing, screening and beyond”, Nature methods, vol. 15, no. 11, pp. 871-879.
Kester, L. & van Oudenaarden, A. 2018, “Single-cell transcriptomics meets lineage tracing”, Cell Stem Cell, vol. 23, no. 2, pp. 166-179.
Yin, H., Kanasty, R. L., Eltoukhy, A. A., Vegas, A. J., Dorkin, J. R. & Anderson, D. G. 2014, “Non-viral vectors for gene-based therapy”, Nature Reviews Genetics, vol. 15, no. 8, pp. 541-555.
Gao, Y., Huang, J., O'Keeffe Ahern, J., Cutlar, L., Zhou, D., Lin, F. & Wang, W. 2016, “Highly Branched Poly (β-amino esters) for Non-Viral Gene Delivery: High Transfection Efficiency and Low Toxicity Achieved by Increasing Molecular Weight”, Biomacromolecules, vol. 17, no. 11, pp. 3640-3647.
Kim, H. J., Kim, A., Miyata, K. & Kataoka, K. 2016, “Recent progress in development of siRNA delivery vehicles for cancer therapy”, Advanced Drug Delivery Reviews, vol. 104, pp. 61-77.
Wang, H., Jiang, Y., Peng, H., Chen, Y., Zhu, P. & Huang, Y. 2015, “Recent progress in microRNA delivery for cancer therapy by non-viral synthetic vectors”, Advanced Drug Delivery Reviews, vol. 81, pp. 142-160.
Dunn, A. W., Kalinichenko, V. V. & Shi, D. “Highly Efficient In Vivo Targeting of the Pulmonary Endothelium Using Novel Modifications of Polyethylenimine: An Importance of Charge”, Advanced healthcare materials, pp. 1800876.
Dahlman, J. E., Kauffman, K. J., Xing, Y., Shaw, T. E., Mir, F. F., Dlott, C. C., Langer, R, Anderson, D. G. & Wang, E. T. 2017, “Barcoded nanoparticles for high throughput in vivo discovery of targeted therapeutics”, Proceedings of the National Academy of Sciences, vol. 114, no. 8, pp. 2060-2065.
Paunovska, K., Gil, C. J., Lokugamage, M. P., Sago, C. D., Sato, M., Lando, G. N., Gamboa Castro, M., Bryksin, A. V. & Dahlman, J. E. 2018, “Analyzing 2000 in vivo drug delivery data points reveals cholesterol structure impacts nanoparticle delivery”, ACS nano, vol. 12, no. 8, pp. 8341-8349.
Marino, G., Niso-Santano, M., Baehrecke, E. H. & Kroemer, G. 2014, “Self-consumption: the interplay of autophagy and apoptosis”, Nature reviews Molecular cell biology, vol. 15, no. 2, pp. 81-94.
Kale, J., Osterlund, E. J. & Andrews, D. W. 2018, “BCL-2 family proteins: changing partners in the dance towards death”, Cell Death & Differentiation, vol. 25, no. 1, pp. 65-80.
Koike, H., Iwasawa, K., Ouchi, R, Maezawa, M., Giesbrecht, K., Saiki, N., Ferguson, A., Kimura, M., Thompson, W. L. & Wells, J. M. 2019, “Modelling human hepato-biliary-pancreatic organogenesis from the foregut-midgut boundary”, Nature, vol. 574, no. 7776, pp. 112-116.
Ouchi, R., Togo, S., Kimura, M., Shinozawa, T., Koido, M., Koike, H., Thompson, W., Karns, R. A., Mayhew, C. N. & McGrath, P. S. 2019, “Modeling steatohepatitis in humans with pluripotent stem cell-derived organoids”, Cell metabolism, vol. 30, no. 2, pp. 374-384. e6.
MacParland, S. A., Liu, J. C., Ma, X., Innes, B. T., Bartczak, A. M., Gage, B. K., Manuel, J., Khuu, N., Echeverri, J. & Linares, I. 2018, “Single cell RNA sequencing of human liver reveals distinct intrahepatic macrophage populations”, Nature communications, vol. 9, no. 1, pp. 1-21.
Luzio, J. P., Pryor, P. R. & Bright, N. A. 2007, “Lysosomes: fusion and function”, Nature reviews Molecular cell biology, vol. 8, no. 8, pp. 622-632.
Mayor, S. & Pagano, R. E. 2007, “Pathways of clathrin-independent endocytosis”, Nature reviews Molecular cell biology, vol. 8, no. 8, pp. 603.
McMahon, H. T. & Boucrot, E. 2011, “Molecular mechanism and physiological functions of clathrin-mediated endocytosis”, Nature reviews Molecular cell biology, vol. 12, no. 8, pp. 517.

What is claimed is:

1. A method of synthesizing a capped cationic polymer, comprising:
(a) contacting poly(ethylene glycol) diacrylate monomers and 3-amino-1-propanol to form a poly(ethylene glycol) diacrylate/3-amino-1-propanol cationic polymer by Michael Addition, wherein the molar ratio of poly(ethylene glycol) diacrylate monomers to 3-amino-1-propanol is greater than 1, and wherein the cationic polymer is acrylate terminated;
(b) contacting the terminal acrylate groups of the cationic polymer with capping molecules comprising amine groups to form the capped cationic polymer by Michael Addition, wherein the capped cationic polymer does not comprise any acrylate groups.

2. The method of claim 1, wherein the poly(ethylene glycol) diacrylate monomers and 3-amino-1-propanol of step (a) are further contacted with di(trimethylolpropane) tetraacrylate, wherein the addition of di(trimethylolpropane) tetraacrylate results in the formation of a branched poly(ethylene glycol) diacrylate/di(trimethylolpropane) tetraacrylate/3-amino-1-propanol cationic polymer comprising more than two terminal acrylate groups.

3. The method of claim 1 or 2, wherein the capping molecules comprise one or more of 1,4-bis(3-aminopropyl)piperazine, spermine, polyethylenimine, or 2,2-dimethyl-1,3-propanediamine, or any combination thereof.

4. The method of any one of the preceding claims, wherein the molar ratio of poly(ethylene glycol) diacrylate monomers to 3-amino-1-propanol is 1.01:1, 1.02:1, 1.03:1, 1.04:1, 1.05:1, 1.06:1, 1.07:1, 1.08:1, 1.09:1, 1.1:1, 1.11:1, 1.12:1, 1.13:1, 1.14:1, or 1.15:1, or about 1.01:1, about 1.02:1, about 1.03:1, about 1.04:1, about 1.05:1, about 1.06:1, about 1.07:1, about 1.08:1, about 1.09:1, about 1.1:1, about 1.11:1, about 1.12:1, about 1.13:1, about 1.14:1, or about 1.15:1, or any ratio within a range defined by any two of the aforementioned ratios, for example, 1.01:1 to 1.15:1, 1.01:1 to 1.1:1, 1.05:1 to 1.1:1, or 1.1:1 to 1.15:1.

5. The method of any one of the preceding claims, wherein the mass ratio of the cationic polymer and the capping molecules is 100:1, 100:2, 100:3, 100:4, 100:5, 100:6, 100:7, 100:8, 100:9, 100:10, 100:15, 100:20, 100:25, 100:30, 100:35, 100:40, 100:45, 100:50, 100:55, 100:60, 100:65, 100:70, 100:75, 100:80, 100:85, 100:90, 100:95, 100:100, 100:150, 100:200, 100:300, 100:400, or 100:500, or about 100:1, about 100:2, about 100:3, about 100:4, about 100:5, about 100:6, about 100:7, about 100:8, about 100:9, about 100:10, about 100:15, about 100:20, about 100:25, about 100:30, about 100:35, about 100:40, about 100:45, about 100:50, about 100:55, about 100:60, about 100:65, about 100:70, about 100:75, about 100:80, about 100:85, about 100:90, about 100:95, about 100:100, about 100:150, about 100:200, about 100:300, about 100:400, or about 100:500, or any ratio within a range defined by any two of the aforementioned ratios, for example, 100:1 to 100:500, 100:1 to 100:25, 100:10 to 100:100, or 100:100 to 100:500.

6. The method of any one of the preceding claims, wherein the capped cationic polymer is POLY1, POLY2, POLY3, POLY4, POLY5, POLY6, POLY7, or POLY8, or any combination thereof.

7. The method of any one of the preceding claims, wherein the cationic polymers and capped cationic polymers are synthesized according to the ratios and components shown in Table 2.

8. The capped cationic polymer synthesized by the method of any one of claims 1-3.

9. The capped cationic polymer of any one of the preceding claims, further comprising a fluorescent dye.

10. The capped cationic polymer of claim 9, wherein the fluorescent dye is DyLight 488, DyLight 550, or DyLight 650.

11. A method of labeling a cell, comprising contacting the cell with a cationic barcode, wherein the cationic barcode comprises a cationic polymer and a nucleic acid barcode, wherein the cationic polymer permits the nucleic acid barcode to access the cytoplasm of the cell.

12. The method of claim 11, wherein the nucleic acid is DNA or RNA.

13. The method of claim 11 or 12, wherein the nucleic acid is single stranded DNA (ssDNA).

14. The method of any one of claims 11-13, wherein the nucleic acid has a length of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, or 5000 nucleotides in length, or any length within a range defined by any two of the aforementioned lengths, for example, 10 to 5000 nucleotides, 100 to 1000 nucleotides, 200 to 500 nucleotides, 10 to 500 nucleotides, or 400 to 5000 nucleotides in length.

15. The method of any one of claims 11-14, wherein the cationic polymer is the capped cationic polymer of the method of any one of claims 1-10.

16. The method of any one of claims 11-15, wherein the cell is part of a tissue, organoid, or spheroid, or any combination thereof.

17. The method of claim 16, wherein the cell is part of a liver organoid or a foregut spheroid.

18. The method of any one of claims 11-17, wherein the nucleic acid has the sequence of SEQ ID NO: 2-4.

19. A method of multiplexed barcoding of a population of cells, comprising:
contacting the population of cells with one or more cationic barcodes, wherein each of the cationic barcodes comprises a cationic polymer and a nucleic acid barcode of a unique sequence; and
sequencing the nucleic acid barcodes of the one or more cationic barcodes by single cell RNA-seq, thereby identifying individual cells as belonging to the population of cells by the sequences of the nucleic acid barcodes of the individual cells.

20. The method of claim 19, wherein the cationic polymer is the capped cationic polymer of the method of any one of claims 1-10.

21. The method of claim 19 or 20, wherein the nucleic acid barcode is a ssDNA barcode and sequencing the nucleic acid barcodes comprises amplifying the ssDNA barcode.

22. The method of any one of claims 19-21, wherein the nucleic acid barcode has the sequence of SEQ ID NO: 2-4.

23. The method of any one of claims 19-22, wherein the population of cells is part of a tissue, organoid, or spheroid.

24. The method of claim 23, wherein the population of cells is part of a liver organoid or a foregut spheroid.

25. The method of any one of claims 19-24, wherein the population of cells comprises two or more subpopulations of cells, wherein each subpopulation of cells is from a unique individual and the population of cells is formed by combining the two or more subpopulations of cells.

26. The method of claim 25, wherein contacting the population of cells comprises contacting each of the two or more subpopulations of cells with a unique cationic barcode before the population of cells is formed by combining the two or more subpopulations of cells.

27. The method of claim 26, wherein sequencing comprises sequencing the unique cationic barcode of each of the two or more subpopulations of cells, thereby identifying individual cells as belonging to one of the two or more subpopulations of cells by the sequences of the nucleic acid barcodes of the individual cells.


SEQUENCE LISTING (4 sequences)

SEQ ID NO: 1 - DNA, 25 bp, Artificial Sequence
  Barcode amplification primer
  3′_phosphorothioate (24)..(25)
gtgactggagttcagacgtgtgctc

SEQ ID NO: 2 - DNA, 77 bp, Artificial Sequence
  Barcode E2
gtgactggagttcagacgtgtgctcttccgatctcatcttgtgatcbaaaaaaaaaaaaaaaaaaaaaaaaaaaaaa

SEQ ID NO: 3 - DNA, 77 bp, Artificial Sequence
  Barcode E3
gtgactggagttcagacgtgtgctcttccgatctagaaggacgagtbaaaaaaaaaaaaaaaaaaaaaaaaaaaaaa

SEQ ID NO: 4 - DNA, 77 bp, Artificial Sequence
  Barcode E4
gtgactggagttcagacgtgtgctcttccgatctcaccatgtaccabaaaaaaaaaaaaaaaaaaaaaaaaaaaaaa